(19) United States
(12) Patent Application Publication
(10) Pub. No.: US 2026/0219174 A1
(43) Pub. Date: Jul. 30, 2026

(54) METHODS FOR DIAGNOSING OR MONITORING A DISEASE IN A SUBJECT USING SPECTROSCOPY

(71) Applicants: King Abdullah University of Science and Technology, Thuwal (SA); King Saud University, Riyadh (SA)

(72) Inventors: Roberto INCITTI, Thuwal (SA); Takashi GOJOBORI, Thuwal (SA); Carlo LIBERALE, Thuwal (SA); Jean-Marc Andre NABHOLTZ, Riyadh (SA); Khalid AL-SALEH, Riyadh (SA); Elisa GRASSI, Thuwal (SA); Mohun R.K. BAHADOOR, Beaumont (FR)

(21) Appl. No.: 19/145,913

(22) PCT Filed: Dec. 22, 2023
(86) PCT No.: PCT/IB2023/063176

§ 371 Date: Jul. 4, 2025

Related U.S. Application Data
(60) Provisional application No. 63/435,371, filed on Dec. 27, 2022.

(51) Int. Cl. G01N 21/35; G01N 21/65

(52) U.S. Cl. CPC G01N 21/35; G01N 21/65; G01N 2201/129

(57) ABSTRACT

A method for diagnosing a disease, such as breast cancer, in a biological sample using spectroscopic data is described. The method involves a computer-implemented method that converts spectroscopic vibrational from the sample into a profile and scores the profile using a pair of reference profiles. Based on the score and a threshold, it can be determined whether the subject from which the sample was obtained has a disease, and, if so, to what extent. The method also allows detection of early and pre-disease states in subjects based on the detection of the signal of low concentration analytes that are indicative of early or incipient disease states. The method is non-invasive, non-subjective, highly specific, and sensitive. The method affords the application of a single standard of diagnostic accuracy, independent of the availability of expert pathologists.

### FIELD

This invention is generally related to the assessment or monitoring of a disease in a subject, more specifically by assaying biological samples from a subject using spectroscopy.

### BACKGROUND

Over the last five to six decades, there have been numerous efforts to improve upon the ability to assay biological samples to diagnose diseases in humans and other animals, and devise effective treatment plans to manage or to eliminate diseases. Efforts have also been expended to develop the capacity to follow the evolution of a disease from the earliest detectable time, to determine the most opportune time to intervene therapeutically and to choose for each patient the most efficient and effective form of therapy.

Despite all these efforts, the available methods for evaluating biological samples still lack large-scale applicability in a clinical setting, and lack the ability to manageably monitor, in real-time, the effect of a treatment regimen on the evolution of a disease, such as cancer. As such, there are few applicable solutions in the clinic, from early stages, such as at a precancerous stage, as well as in the later stages, where “real-time” evaluation of the rate of progression or regression of the disease in each patient, performed by accurate methods comparing the properties of samples taken at different times/stages, trigger “manageable” actions that could be implemented at reasonable overall costs.

Current methods used in the clinic cannot accurately grade the extent of progress of precancerous disease in an individual patient. The course and the response of the disease to therapy are only accessible via retrospective epidemiological studies that give, at best, the average course of a disease and the average response of that disease to treatment.

Another problem with current diagnostic methods involves subjectivity, often manifested in the poor agreement between the conclusions of different pathologists examining the same data set. This subjectivity is particularly acute in diagnostic methods that involve microscopic viewing of samples. What complicates the diagnosis is that not all cells in a section of tissue or on a slide are affected equally if affected at all. In addition, extensive changes in the chemical and physical attributes at the molecular level in cells may not appear due to changes in the morphology of the cells in the preanalytical pathology phase. Further, and especially in the context of early diagnosis, the pathologist is often looking for a few diseased cells amongst a large number of normal-appearing cells. Thus, a relationship exists between the validity of a diagnostic conclusion and the skill, diligence, and prior experience of the pathologist. It is difficult to control these variations so long as the fundamental method of diagnostic pathology remains a subjective process.

Moreover, many of the existing methods for obtaining samples involve invasive and sometimes cumbersome procedures that include but are not limited to fine needle aspirations, macro-biopsies, sentinel lymph node dissection, and sometimes upfront breast surgeries.

An objective of all new cancer diagnostics is to develop a test that can detect the disease at very early stages, with less invasiveness, lower costs and higher specificity. At present, available cancer diagnostic tests can be classified into four categories: cytology-based tests, that provide cell-based evidence of abnormal cancerous cells; imaging tests, which are scan-based tests that provide visual evidence of the disease (for example ultrasound for ovarian cancer, Basic Specific Gamma Imaging (BSGI) tests for breast cancer (BC)); biological tests that measure biological factors associated with the disease (for example HER-2 test for BC); and genomic tests which display and measure genetic predisposition of individuals for cancer, or analyze the activity of a group of genes that can affect how a cancer is likely to behave and respond to treatment (for example Oncotype-DX assay in BC).

Tumors with similar histopathological appearances can follow significantly different clinical courses and show different responses to therapy. In a few cases, such clinical heterogeneity has been explained by dividing morphologically similar tumors into subtypes with distinct pathogens. For example, BC is classified into 5 distinctive subtypes, colorectal cancer in 7 and up to 20 descriptions for lung cancer are noted in the scientific literature.

One development in the diagnosis of cancer is the identification of biological markers. These markers are biological molecules that appear due to the advent of the disease. At times these molecules are found in elevated levels which may be correlated with the disease.

Another development to improve the assessment of BC diagnostics is immunohistochemistry (IHC) testing. IHC involves using specific antibodies to detect the expression and expression levels of known biomarkers in biological samples. However, IHC has not sufficiently addressed the needs of the diagnostic industry owing to its requirements for accurate labeling, tagging, and specific knowledge of molecular biomarkers expressed in a disease, as well as the cumbersome production of antibodies that target those molecular biomarkers.

In cancer pathology laboratories, quality may also be an issue. The consequences of wrongful testing can range from minimal (assignment of normal results to unrelated normal individuals) to disastrous (administration of incompatible treatment). Generally, there are three kinds of errors associated with clinical laboratory testing: pre-analytical, analytical, and post-analytical.

Pre-analytical errors occur before the sample is tested. The vast majority occur even before the sample arrives in the laboratory and may not involve lab personnel. Pre-analytical errors include patient misidentification, inappropriate patient preparation, collection of an inappropriate sample, collection of an appropriate sample in an inappropriate receptacle, and sample degradation due to mistreatment, such as overheating, freezing or delayed transport. Pre-analytical errors are among the most common errors associated with clinical laboratories testing. One reason for these errors is that the steps involved in sample collection have not been standardized or uniformly accepted by all those involved in the process, such as physicians, nurses, healthcare trainees, patient care technicians, or clerical staff. Scarcity and the quality of these resources are some of the other possible origins of pre-analytical errors.

Analytical errors may occur after receipt of the sample by the laboratory, sometimes during the analytical phase of testing. They constitute a relatively smaller percentage of the errors, because much of the testing in today's clinical laboratory is automated. Errors in the analytical phase, which may be attributed to the laboratory personnel, often involve missteps in specimen processing and storage, unrecognized quality control failures, instruments miscalibration, instrument misuse, procedural deviations and errors, interfering substances, and use of inappropriate or expired reagents. Laboratories may be able to control the analytical phase of testing by strict adherence to standard operating procedures, attentiveness to quality control outcomes, regular preventative maintenance of instrumentation, and scheduled proficiency testing. Although relatively few, analytical errors can be vexing, often coming to light only after the laboratory have been notified that a finalized result is inconsistent with a patient's condition, status, or treatment.

Post-analytical errors refer to errors made during the reporting and/or interpretation of test results. These errors often arise from laboratory personnel, or individuals who order the tests. Post analytical errors may occur more frequently than analytical errors, but less frequently than pre-analytical ones. Failure to post results, result entry errors (especially for tests recorded manually), may result in misassignment, inappropriate test utilization, and slow turnaround time.

Other attempted solutions to improve cancer assessments involving optical spectroscopic techniques to analyze biological samples are detailed below.

The diagnostic pathology services that are used to perform these analyses have inherent limitations. For example, the methods often require knowledge of pre-existing associations between peaks, shapes of peaks, various wavelengths, and specific organs to produce a meaningful diagnosis. Further complicating the analysis can be a requirement of the knowledge of different vibrational modes of biological molecules. Accordingly, these methods often require interpretation and/or analysis of spectrograms by expert pathologists, which in turn makes it difficult to provide high-quality diagnostic pathology services in medically underserved regions of the world. As such, these services may not be available in the absence of trained pathologists in reasonable proximity to sites at which biological samples are collected. Therefore, when such services are performed without the assistance of trained pathologists, the quality of these services may be extremely poor.

Another limitation involves spectroscopic techniques with poor specificity and sensitivity. These spectroscopic techniques probe substructures present in molecules but may not probe entire molecules. However, the occurrence of the same substructures in different molecules may cause overlaps in the spectral responses, limiting the identification of specific molecules in complex samples. Other spectroscopic techniques are often limited by their inability to reliably detect the presence of components that account for less than 5% by weight of the total mass of the sample.

Accordingly, there remains an unmet need to develop non-invasive, non-subjective, highly specific, and/or sensitive methods for the screening, diagnosis, and/or prognosis of diseases in humans or animals.

### SUMMARY

Disclosed herein are non-invasive, non-subjective, quantitative systems and methods for analyzing biological samples and making interpretations about the presence or absence of a disease, and optionally, if present, the stage, or grade level of the disease.

A method for screening, diagnosis, and/or prognosis of BC in a subject using molecular biomarkers in the subject's sample, is described. The subject can be a human or other animal, and the method may be performed for example in vitro. The method combines an optical spectroscopic technique and a computer-implemented technique.

For example:

The optical spectroscopic technique is carried out using a Raman spectrometer with a 785 nm-wavelength laser and measuring, for example, a vibrational frequency span between 3100 cm⁻¹ and 900 cm⁻¹, such as 3050.855 cm⁻¹ and 929.527 cm⁻¹, inclusive, and 1101 points. This number of points and the vibrational frequency span are determined by the chosen range of the spectral frequencies and the spectral resolution of the instrument.

Also described are methods of using the methods described herein. The methods can be used for the screening, diagnosis, and/or prognosis of BC.

In one aspect of the invention, there is provided a method of diagnosing or monitoring a disease in a subject, the method comprising:
1) providing or obtaining a sample from the subject;
2) generating a spectroscopic profile of the sample;
3) comparing the spectroscopic profile to one or more reference spectroscopic profiles to obtain a general score;
4) determining a status of the disease by comparing the general score to a threshold value.

In a further aspect of the method or methods outlined above, generating the spectroscopic profile comprises:
obtaining a raw vector, Vr, the raw vector comprising a two-dimensional vector of a plurality of wave numbers, with each intensity value corresponding to a wave number;
filtering the raw vector; and
normalizing the plurality of intensity values of the raw vector to generate a normalized vector, Vn.

In a further aspect of the method or methods outlined above, the filtering comprises smoothing the raw vector and removing background noise.

In a further aspect of the method or methods outlined above, the spectroscopic profile is further generated by selecting a subset vector (Vf), of the normalized vector, Vn.

In a further aspect of the method or methods outlined above, the subset vector comprises, for example, between 6 and 30 components.

In a further aspect of the method or methods outlined above, the wave numbers of the subset vector are in non-overlapping ranges of between 20 to 60 cm⁻¹ in width.

In a further aspect of the method or methods outlined above, the general score is determined by a polynomial model of a degree between 1 and 6 computed on the subset vector and whose coefficients are determined on a learning set.

In a further aspect of the method or methods outlined above, the general score is compared to the threshold value.

In a further aspect of the method or methods outlined above, the threshold value is tuned to a higher value for diagnosing the disease or a lower value for monitoring the disease.

In a further aspect of the method or methods outlined above, Vf comprises one or more values within a range of about 300 to about 1900 cm⁻¹.

In a further aspect of the method or methods outlined above, Vf comprises four values between about 620 to about 670 cm⁻¹, two values between about 720 to about 760 cm⁻¹, two values between about 1550 to about 1580 cm⁻¹, and four values between about 1740 to about 1790 cm⁻¹.

In a further aspect of the method or methods outlined above, the spectroscopic profile is generated via vibrational spectroscopy, field-resolved spectroscopy, frequency-resolved spectroscopy, Fourier-transform infrared spectroscopy, Raman spectroscopy, infrared attenuated total reflectance, diffuse reflectance spectroscopy, or combinations thereof.

In a further aspect of the method or methods outlined above, field-resolved spectroscopy comprises field-resolved infrared spectroscopy.

In a further aspect of the method or methods outlined above, vibrational spectroscopy comprises infrared spectroscopy, such as near-infrared spectroscopy, mid-infrared, and/or far-infrared.

In a further aspect of the method or methods outlined above, the spectroscopic profile is generated via Raman spectroscopy.

In a further aspect of the method or methods outlined above, the spectroscopic profile is measured between about 15,000 cm⁻¹ to about 200 cm⁻¹.

In a further aspect of the method or methods outlined above, at least one of the one or more reference spectroscopic profiles is generated using a sample from a non-diseased subject.

In a further aspect of the method or methods outlined above, at least one of the one or more reference spectroscopic profiles is generated using a sample from a diseased subject.

In a further aspect of the method or methods outlined above, at least one of the one or more reference spectroscopic profiles is generated using a cancerous sample.

In a further aspect of the method or methods outlined above, the disease is cancer.

In a further aspect of the method or methods outlined above, the cancer is breast cancer or colon cancer.

In a further aspect of the method or methods outlined above, the status is a TNM stage.

In a further aspect of the method or methods outlined above, the method or methods further comprise treating the subject for the disease.

In a further aspect of the method or methods outlined above, at least one of the one or more reference spectroscopic profiles is from one or more individuals in the same population as the subject.

In a further aspect of the method or methods outlined above, all the reference spectroscopic profiles are from one or more individuals in the same population as the subject.

In a further aspect of the method or methods outlined above, at least one of the one or more reference spectroscopic profiles is from one or more individuals in a different population than the subject.

In a further aspect of the method or methods outlined above, all the reference spectroscopic profiles are from one or more individuals in a different population than the subject.

In a further aspect of the method or methods outlined above, the one or more reference spectroscopic profiles is from the subject.

In a further aspect of the method or methods outlined above, the subject is a human.

In a further aspect of the method or methods outlined above, the sample is in vitro.

In a further aspect of the method or methods outlined above, the sample comprises blood, spittle/saliva, serum, plasma, urine, sputum, sweat, semen, synovial fluids, lymphatic fluids, cerebrospinal fluids, biopsy, stool, or combinations thereof.

In a further aspect of the method or methods outlined above, the subject is asymptomatic of the disease.

In a further aspect of the method or methods outlined above, the subject is presenting symptoms of the disease.

In a further aspect of the method or methods outlined above, the subject has not had or has a prior history of having cancer.

In a further aspect of the method or methods outlined above, the subject exhibits one or more symptoms selected from the group consisting of breast pains, breast nodules, nipple discharge, weight loss, fatigue, anemia, and a combination thereof.

In a further aspect of the method or methods outlined above, the subject is at risk of developing breast cancer.

In a further aspect of the method or methods outlined above, the subject is exposed to one or more assays for identification of breast cancer.

### BRIEF DESCRIPTION OF THE DRAWINGS

The drawings included herewith are for illustrating various examples of articles, methods, and systems of the present specification and are not intended to limit the scope of what is taught in any way. In the drawings:

FIG. 1 is a schematic diagram for performing an embodiment of the methods described herein; and

FIG. 2 is a flow chart diagram of an example method of diagnosing a disease in a subject in accordance with at least some embodiments.

### DETAILED DESCRIPTION

### I. Definitions

“Non-subjective,” as used herein relating to screening, diagnosis, and/or prognosis, will be understood as visual inspection of a sample and/or analysis of a spectrogram is not required to determine whether the sample is collected from a diseased or non-diseased patient.

### II. Method for Screening, Diagnosis, and/or Prognosis of Diseases

The subject can be human or other animals, using molecular biomarkers in the subject's sample. The method may be used on a sample for example in vitro. Preferably, the method is non-invasive. Currently, knowledge of each specific molecular biomarker is not required. The method can integrate all molecular biomarkers of profiles (which may be unique for a given subject at a given time) and correlate the results to a given question, which can be in a binary mode, such as existence or non-existence of BC. An inquiry can also be along the lines of assessing the stage (grade level) of BC if BC is detected. Accordingly, the method offers a significant improvement, because it provides a user with an elegant and streamlined quantitative analysis tool to probe a sample containing multiple molecules within a complex environment and to arrive at a diagnosis and/or prognosis without the need for (i) the user's knowledge of disease-specific molecular biomarkers in the sample and/or (ii) expertise in the interpretation of spectrograms. In some forms, knowledge of specific molecular biomarkers can be useful to link molecular profiles to specific biological modifications related to the asked binary question.

In some cases, the method involves an analytical method, a computer-implemented method, or a combination thereof. In some forms, the method involves both an analytical method and a computer-implemented method.

In some forms, the method involves (i) generating a spectroscopic profile of a subject's sample using an analytical method, such that the spectroscopic profile contains one or more components, (ii) obtaining a general score of the spectroscopic profile using a computer-implemented technique, and/or (iii) providing a diagnosis, prognosis, or both, of the disease based on the general score.

In some forms, computing the general score involves using all the components of the spectroscopic profile. In other forms, computing the general score involves using some of the components of the spectroscopic profile.

In some forms, the method involves screening and diagnosis of BC by performing a RAMAN measurement of a human sample, in particular those obtained in a non-invasive way, for example, using blood and computing a score based on the whole set or from a part of the RAMAN measurement.

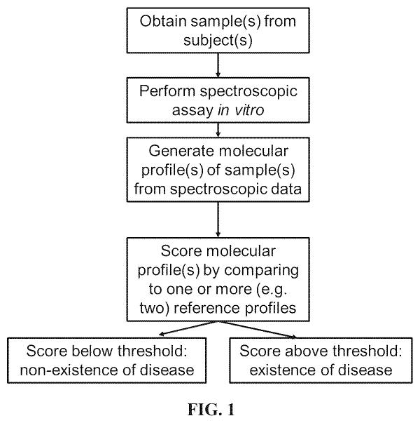

Referring to FIG. 1, the analytical method (such as spectroscopic assay) can be performed in vitro. For a given sample, the method includes generating a spectroscopic profile containing data (such as vibrational frequencies, or measured intensities at specific vibrational frequencies) of the sample based on the spectroscopic assay; assigning a score to that profile by comparing, preferably, to a set of reference profiles containing data (such as vibrational frequencies, or measured intensities at specific vibrational frequencies), and/or determining by the score whether the subject from which the sample was obtained has a disease, and optionally, if present, at what stage (grade level).

In some forms, an analytical method (such as one described herein) involves a spectroscopic instrument, implements a spectroscopic technique, such as optical spectroscopy. In some forms, the spectroscopic instrument can be a Raman spectrometer with a frequency span between 3100 cm⁻¹ and 900 cm⁻¹, such as 3050.855 cm⁻¹ and 929.527 cm⁻¹, inclusive, and 1101 points.

In some forms, a computer-implemented technique (such as one described herein) can be used to determine a patient's disease status based on the data obtained from a spectroscopic instrument. Preferably, the spectroscopic data is a function of the molecular profile of the sample being analyzed.

Preferably, in some forms, the components of the spectroscopic profile of the sample and those in the one or more reference spectroscopic profiles contain vibrational frequencies or measured data at specific vibrational frequencies. In some forms, the component scores can be summed to obtain a general score. In some forms, when the general score is greater than a threshold, the subject is deemed to have a disease, otherwise, the subject is disease-free. The general score is used to determine whether the subject has BC.

In some forms, at least one of the one or more reference spectroscopic profiles are generated using a sample from a non-diseased patient. In some forms, at least one of the one or more reference spectroscopic profiles are generated by using a diseased sample. In some forms, at least one or more reference spectroscopic profiles is generated using a cancerous sample. In some forms, the cancerous sample has BC.

The method can involve a probability in the screening, diagnosis, and/or prognosis, where a limited number of factors are used. For instance, for BC, where a limited number of factors are used for classifications: clinical-stage, hormonal receptors (estrogen and progesterone), amplification of the HER-2 gene, and cell proliferation (mitotic index or Ki-67), this can lead to the definition of large subgroups, which are heterogeneous by nature, as BC, on an individual patient basis, can be more complex than that. Spectroscopy by Raman, used here, allows to apprehend a partial molecular reality (the spectral results are reproducible for a given sampling) with a much wider array of biomarkers, some being specific to the disease or its biological consequences, some being specific of the host. Their computational integration, through batteries of tests, allows for the differentiation of individual profiles when asking relevant questions in a binary mode (more holistic approach).

i. Analytical Methods

The analytical methods include one or more spectroscopic techniques. Examples of spectroscopic techniques include, but are not limited to, field-resolved spectroscopy (such as field-resolved infrared spectroscopy), frequency-resolved spectroscopy, Fourier-transform infrared spectroscopy, Raman spectroscopy, infrared attenuated total reflectance, diffuse reflectance spectroscopy, and combinations thereof.

In some preferred forms, the spectroscopic technique involves time- or frequency-resolved spectroscopy.

In some forms, the spectroscopic technique involves vibrational spectroscopy. In some forms, vibrational spectroscopy includes infrared spectroscopy, such as near-infrared spectroscopy, mid-infrared, far-infrared, or Raman spectroscopy.

In some instances, spectroscopic methods probe the chemical substructures present in molecules, not entire molecules by detecting vibrational responses to infrared or Raman excitation.

The spectroscopic instrument can be operated over a range of vibrational frequencies. The frequency range can span between but is not limited to, about 14,000 cm⁻¹ and about 800 cm⁻¹, and sub-ranges within it. In some forms, the spectroscopic instrument can be a broadband femtosecond resolved broadband infrared laser source, coupled with an infrared wave sampling system for ultra-sensitive molecular vibration spectroscopy. In some forms, the frequency scan ranges between about 3050.855 cm⁻¹ and about 929.527 cm⁻¹. In some forms, the spectroscopic instrument can be a Raman spectrometer with a frequency span between 3050.855 cm⁻¹ and 929.527 cm⁻¹, inclusive, and 1101 points.

Preferably, the spectroscopic instrument uses high resolution. High spectral resolution can include spectral sampling between 1 cm⁻¹ and 10 cm⁻¹, such as 1 cm⁻¹, 2 cm⁻¹, 3 cm⁻¹, 4 cm⁻¹, 5 cm⁻¹, 6 cm⁻¹, 7 cm⁻¹, 9 cm⁻¹, 9 cm⁻¹, or 10 cm⁻¹.

ii. Computer-Implemented Method

The computer-implemented method described herein is not limited to any particular spectroscopic analytical technique. The computer-implemented method implements an approach that is capable of general spectroscopic profiles using data generated from field-resolved spectroscopy (such as field-resolved infrared spectroscopy), frequency-resolved spectroscopy, Fourier-transform infrared spectroscopy, Raman spectroscopy, infrared attenuated total reflectance, diffuse reflectance spectroscopy, and combinations thereof.

The computer-implemented method can be performed on a computer that is capable of executing computer program instructions to carry out the approach. For example, the computer may have both volatile and non-volatile memory (the latter for initially storing the computer program instructions), input and output devices such as keyboard and a display, and one or more processors such as a central processing unit (CPU), graphics processing unit (GPU), and so forth. The computer can be in physical proximity to the spectroscopic instrument that generates the data. The computer can also be at a remote location and is connected to the spectroscopic instrument via ethernet, Bluetooth, near field communication, Wi-Fi, integrated circuits, or a combination thereof. Non-limiting examples of the processes performed by the technique are described in the Example. Briefly, the technique generates a spectroscopic profile containing one or more components, which is reflective of the molecular profile of the sample.

In one non-limiting example, the spectroscopic profile contains 1,101 features, determined from (3050.856 cm⁻¹-925.547 cm⁻¹)/(spectral sampling (2 cm⁻¹)). The feature at each position in the spectroscopic profile corresponds to photon count intensity at that wavenumber. Accordingly, the length of the spectroscopic profile can be any value but limited by the span of the frequency range and the spectral sampling of the instrument.

Generally, the raw data obtained through measurement consists of a two-dimensional vector of, respectively, a wave number and an intensity value. Further, the vector obtained from the raw measurement, Vr, is processed using successive steps of (i) raw data smoothing; (ii) removal of background; (iii) normalization; (iv) determining a class between diseased and not diseased.

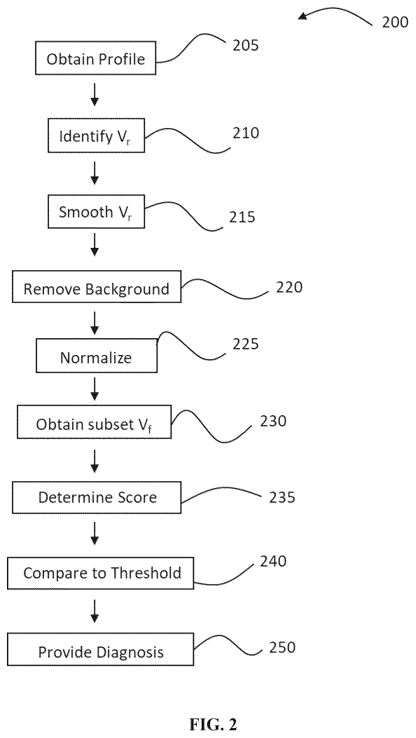

Referring now to FIG. 2, there is illustrated an example method of diagnosing a disease in a subject in accordance with at least some embodiments. Method 200 may be carried out by a computer, as described herein.

At block 205, a spectroscopic profile is obtained, as described herein; the components of the spectroscopic profile of the subject's sample contain vibrational frequencies (as do those of reference profiles). For example, the spectroscopic profile of the subject's sample, and one or more reference profiles may be generated using data from a spectroscopic technique that applies a frequency scan between, but not limited to, about 14,000 cm⁻¹ and about 800 cm⁻¹, and sub-ranges within it.

The spectroscopic profile of the subject's sample, and one or more reference profiles may be generated using data from a spectroscopic technique comprising field-resolved spectroscopy (such as field-resolved infrared spectroscopy), frequency-resolved spectroscopy, Fourier-transform infrared spectroscopy, Raman spectroscopy, infrared attenuated total reflectance, diffuse reflectance spectroscopy, and combinations thereof.

The spectroscopic technique may involve vibrational spectroscopy, including infrared spectroscopy, such as near-infrared spectroscopy, mid-infrared, and/or far-infrared.

At block 210, a raw vector (e.g., Vr) is identified from the spectroscopic profile, as described elsewhere herein.

At block 215, smoothing may be performed on Vr to generate a smoothed vector Vs. For example, by applying the Savitzky-Golay algorithm for data smoothing, with a polynomial order of 5 and a window of 13.

At block 220, the average spectrum may be calculated from a reference sample. For example, the reference sample may be 6 collected Raman spectra of pure water having the same volume as the plasma blood drops used to generate the spectroscopic profile under analysis. The average spectrum may be used as a reference background spectrum, which can be subtracted or otherwise removed from the smoothed vector Vs to generate a modified smoothed vector. A spectral region is selected for further analysis, such as between about 300 to about 1900 cm⁻¹, or about 600 to about 1800 cm⁻¹. A first mean value of the intensity of the plasma blood spectrum in this spectral region is calculated. A second mean value of the intensity of the background spectrum in the same spectral region is also calculated. Next, the difference between the first and second mean values is calculated and the result is added to the background spectrum, generating a modified background spectrum. Finally, the difference between the plasma blood spectrum and the modified background spectrum is calculated to obtain a smoothed and background-removed vector Vsb.

At block 225, a normalized vector Vn is obtained by dividing all intensity values of Vsb by either the highest value or the lowest value in a top percentile of the intensity values (e.g., the top 1%, 2%, 3%, 4% or 5%).

At block 230, a subset vector Vf of Vn is obtained. The subset vector generally has between 6 and 18 components. For example, in one example, the subset vector consists of 12 wave numbers (wns) and the corresponding intensity values, which may be chosen as follows: 4 wave numbers in a range between 620 and 670 cm⁻¹, 2 wave numbers in a range between 720 and 760 cm⁻¹, 2 wave numbers in a range between 1550 and 1580 cm⁻¹, and 4 wave numbers in a range between 1740 and 1790. In some cases, the subset vector has components within a range of about 300 to about 1900 cm⁻¹. In some cases, the wave numbers of the subset vector are in non-overlapping ranges of between 20 to 60 cm⁻¹ in width.

At block 235, a general score is obtained by computing a polynomial function on Vf of degree 1, 2, 3, 4, 5, 6, whose coefficients are determined by a support vector machine (SVM) on a learning dataset. The score is further used to decide the patient's disease status, or is used to provide a probability of disease, by applying a Platt scaling to the score.

At block 240, the general score is compared to a threshold value or values, to determine the disease/non disease status. For example, the subject may be diagnosed as having a disease when the general score is greater than the threshold value. If the general score includes more than one component, each of the components may be compared with corresponding threshold values.

In some cases, two reference spectra may be used to set upper and lower threshold values. Accordingly, a first reference spectroscopic profile sets upper bounds of spectroscopic data, and a second reference spectroscopic profile sets lower bounds of spectroscopic data.

The threshold values may be determined from one or more reference spectroscopic profiles generated using samples from diseased patients and non-diseased patients. The samples may be labelled a priori using existing methods. For example, the disease may be cancer, such as BC, lung cancer, prostate cancer, colon cancer, skin cancer, blood cancer (leukemia, lymphoma, etc.), myeloma, and a combination thereof.

In some cases, at least one of the reference spectroscopic profiles may be selected from one or more individuals in a same or similar population as the subject, the similarity in population is determined demographically (e.g., similar age, gender, gender, etc.). However, the population need not be the same or similar in all cases.

The diagnosis may be provided at block 250.

In a non-limiting example, the model can be of degree 1 and the disease status of vector Vr will be determined by computing the distance of Vf to the model's separating hyperplane and performing a Platt scaling on the result, then using the value so obtained as a decision threshold to obtain a sensitivity ranging from at least 90% to at least 95%.

In some cases, the status of the disease computed at block 235 is a TNM staging value. A person of skill in the art will understand that the TNM refers to Primary Tumor (T), Regional Lymph Node (N) and Distant Metastasis (M). The TNM value may be determined using the methodology above. In such embodiments, the reference spectroscopic profiles used in the comparative analysis may have a TNM value. Examples of TNM values include: T1NXMX, T2NXMX, T3NXMX and others.

### III. Methods of Using

The methods described herein can be used in the screening, diagnosis, and/or prognosis of BC in humans or other animals.

The sample to be analyzed can include blood samples.

In some forms, the subject is asymptomatic of a disease. In some forms, the subject presents one or more symptoms of a disease. Symptoms include, but are not limited to, breast pains, breast nodules, nipple discharge, weight loss, fatigue, anemia, or a combination thereof. In some forms, the subject has not had or has a prior history of having cancer.

In some forms, the subject is at risk (such as at high risk) of developing BC. In some forms, the subject is exposed to one or more assays for the identification of BC.

A non-limiting example involves using particular patterns of Raman measurements for BC screening and diagnosis. In some forms, the method involves using a combination of particular RS measurement patterns of a variety of molecular biomarkers for BC screening and diagnosis. These molecular biomarkers can be tested in tissue or body fluids (such as blood, serum, plasma, urine, with BC. The format of one RS measurement, termed spectroscopic profile, includes a vector of thousands of variables, each measuring the molecular profile of the bio-fluids at a given time.

Any appropriate method may be used to assess the target directly in the bio-specimen (because the sample preparation step can be skipped in some cases).

In some forms, the method is used as part of a regular checkup. Therefore, in some forms, the subject has not been diagnosed with BC, and, typically for those particular forms, it is not known that a subject has a hyperproliferative disorder, such as a breast neoplasm. In other forms, the individual is at risk for BC, is suspected of having BC, or has a history, personal or family, of cancer, including BC, presence of risk factors such as BRCA1/2 mutations. In some forms, an individual can be known to have cancer and the methods described herein are used to determine the type of BC, stage (grade level), treatment response, and/or prognosis. In some forms, the individual has already been diagnosed with BC and also may be subjected to surgery for BC resection, and/or may undergo methods by the invention to survey the recurrence of BC.

The method also allows detection of early and pre-disease conditions in subjects based on the detection of the signal of low concentration analytes that are indicative of early or incipient disease conditions. For example, the method can be used to detect the presence of abnormalities in samples that are below the level of detection by microscopic and optical spectroscopic examination of samples.

The methods can also be used to determine the stage (grade level) of a diagnosed BC. Further, the computer-implemented methods can be applied to the results of a measure by any high-resolution spectroscopy. The spectroscopic instrument can perform, among others, Fourier-transform infrared spectroscopy, Raman spectroscopy, or any device measuring either infrared intensities or Raman scattering coefficients against vibrational frequencies.

By virtue of the streamlined process of the instant methods, which reduce and/or eliminate subjectivity, the requirement of expert analysis of spectrograms and/or samples under a microscope, the present methods make it possible to provide high-quality detection and/or diagnostic services in medically underserved regions of the world. The methods also provide a basis for immediate diagnostic decisions for patients and physicians, leading in turn to immediate implementation of next-step procedures and treatment. This means that patients and the examining clinician can know almost instantly whether or not the samples examined are from a diseased or a non-diseased patient, and/or the stage (grade level) of disease, if present.

In some forms, the methods can be used to screen and/or diagnose BC at significantly high levels of specificity and sensitivity. In some forms, those high levels can be attributed to the expert medical advice involved in identifying the test data, the advanced spectroscopic technique, and/or the expertise involved in the development and testing of the computer-implemented technique. This level can be much higher than in previously implemented spectroscopic and/or microscopic methods.

Appropriate optical frequencies can be used to probe deeper tissue depths with optical non-invasive methods and the computer-implemented technique is well suited to analyze the output from the probes. The medical importance of this aspect is not simply to allow for gathering immediate diagnostic information from a subject, but also to provide the ability to obtain more information from broader areas by examining samples inside the body than is available by taking biopsies or cells from the body and then examining them. For example, the act per se of biopsy of tissue distorts the remaining tissue, and bleeding that accompanies a biopsy can distort a physician's view of the diseased tissue.

As shown in Table 1 is a proof-of-concept breast cancer (BC) clinical study of several samples obtained from patients and subjected to Raman spectroscopic analysis. Table 1 shows the diagnosis of breast cancer in patients versus control patients, and can be used to train and/or validate the SVM models described herein. As shown in Table 2 is a proof-of-concept colon cancer clinical study of several samples obtained from patients and subjected to Raman spectroscopic analysis. Table 2 shows the diagnosis of colorectal cancer in patients versus control patients, and can be used to train and/or validate the SVM models described herein.

As used herein, the wording “and/or” is intended to represent an inclusive-or. That is, “X and/or Y” is intended to mean X or Y or both, for example. As a further example, “X, Y, and/or Z” is intended to mean X or Y or Z or any combination thereof.

Terms of degree such as “substantially”, “about”, and “approximately” as used herein mean a reasonable amount of deviation of the modified term such that the result is not significantly changed. These terms of degree may also be construed as including a deviation of the modified term if this deviation would not negate the meaning of the term it modifies.

Any recitation of numerical ranges by endpoints herein includes all numbers and fractions subsumed within that range (e.g., 1 to 5 includes 1, 1.5, 2, 2.75, 3, 3.90, 4, and 5). It is also to be understood that all numbers and fractions thereof are presumed to be modified by the term “about” which means a variation of up to a certain amount of the number to which reference is being made if the result is not significantly changed.

The systems and methods described herein may be implemented as a combination of hardware or software. In some cases, the systems and methods described herein may be implemented, at least in part, by using one or more computer programs, executing on one or more programmable devices including at least one processing element, and a data storage element (including volatile and non-volatile memory and/or storage elements). These systems may also have at least one input device (e.g. a pushbutton keyboard, mouse, a touchscreen, and the like), and at least one output device (e.g. a display screen, a printer, a wireless radio, and the like) depending on the nature of the device. Further, in some examples, one or more of the systems and methods described herein may be implemented in or as part of a distributed or cloud-based computing system having multiple computing components distributed across a computing network. For example, the distributed or cloud-based computing system may correspond to a private distributed or cloud-based computing cluster that is associated with an organization.

Some elements that are used to implement at least part of the systems, methods, and devices described herein may be implemented via software that is written in a high-level procedural language such as object-oriented programming language. Accordingly, the program code may be written in any suitable programming language such as Python or Java, for example. Alternatively, or in addition thereto, some of these elements implemented via software may be written in assembly language, machine language or firmware as needed. In either case, the language may be a compiled or interpreted language.

At least some of these software programs may be stored on a storage media (e.g., a computer readable medium such as, but not limited to, read-only memory, magnetic disk, optical disc) or a device that is readable by a general or special purpose programmable device. The software program code, when read by the programmable device, configures the programmable device to operate in a new, specific, and predefined manner to perform at least one of the methods described herein.

Furthermore, at least some of the programs associated with the systems and methods described herein may be capable of being distributed in a computer program product including a computer readable medium that bears computer usable instructions for one or more processors. The medium may be provided in various forms, including non-transitory forms such as, but not limited to, one or more diskettes, compact disks, tapes, chips, and magnetic and electronic storage. Alternatively, the medium may be transitory in nature such as, but not limited to, wire-line transmissions, satellite transmissions, internet transmissions (e.g. downloads), media, digital and analog signals, and the like. The computer usable instructions may also be in various formats, including compiled and non-compiled code.

The disclosed methods can be further understood through the following numbered clauses.

Clause 1. A method of diagnosing or monitoring a disease in a subject, the method comprising:
1) generating a spectroscopic profile of a sample obtained from the subject;
2) comparing the spectroscopic profile to one or more reference spectroscopic profiles to obtain a general score;
3) determining a status of the disease by comparing the general score to a threshold value.

Clause 2. The method of clause 1, wherein generating the spectroscopic profile comprises:
obtaining a raw vector, Vr, the raw vector comprising a two-dimensional vector of a plurality of wave numbers and a plurality of intensity values corresponding to the plurality of wave numbers;
filtering the raw vector; and
normalizing the plurality of intensity values of the raw vector to generate a normalized vector, Vn.

Clause 3. The method of clause 2, wherein the filtering comprises smoothing the raw vector and removing background noise.

Clause 4. The method of clause 2 or clause 3, wherein the spectroscopic profile is further generated by selecting a subset vector (Vf), of the normalized vector, Vn.

Clause 5. The method of clause 4, wherein the subset vector comprises between 6 and 18 components.

Clause 6. The method of clause 5, wherein the wave numbers of the subset vector are in non-overlapping ranges of between 20 to 60 cm⁻¹ in width.

Clause 7. The method of any one of clauses 4 to 6, wherein the general score is determined by a polynomial of a degree between 1 and 6 on the subset vector, the polynomial having been previously determined by a support vector machine on a learning set.

Clause 8. The method of any one of clauses 1-7, wherein the general score is compared to the threshold value.

Clause 9. The method of any one of clauses 1-7, wherein the threshold value is tuned to a higher value for diagnosing the disease or a lower value for monitoring the disease.

Clause 10. The method of clause 5, wherein Vf comprises one or more values within a range of about 300 to about 1900 cm⁻¹.

Clause 11. The method of clause 10, wherein Vf comprises four values between about 620 to about 670 cm⁻¹, two values between about 720 to about 760 cm⁻¹, two values between about 1550 to about 1580 cm⁻¹, and four values between about 1740 to about 1790 cm⁻¹.

Clause 12. The method of any one of clauses 1-11, wherein the spectroscopic profile is generated via vibrational spectroscopy, field-resolved spectroscopy, frequency-resolved spectroscopy, Fourier-transform infrared spectroscopy, Raman spectroscopy, infrared attenuated total reflectance, diffuse reflectance spectroscopy, or combinations thereof.

Clause 13. The method of clause 12, wherein field-resolved spectroscopy comprises field-resolved infrared spectroscopy.

Clause 14. The method of clause 12, wherein vibrational spectroscopy comprises infrared spectroscopy, such as near-infrared spectroscopy, mid-infrared, resonant frequency, and/or far-infrared.

Clause 15. The method of any one of clauses 1-11, wherein the spectroscopic profile is generated via Raman spectroscopy.

Clause 16. The method of any one of clauses 12-15, wherein the spectroscopic profile is measured between about 15,000 cm⁻¹ to about 200 cm⁻¹.

Clause 17. The method of any one of clauses 1 to 16, wherein at least one of the one or more reference spectroscopic profiles is generated using a sample from a non-diseased subject. Clause 18. The method of any one of clauses 1 to 17, wherein at least one of the one or more reference spectroscopic profiles is generated using a sample from a diseased subject. Clause 19. The method of any one of clauses 1 to 17, wherein at least one of the one or more reference spectroscopic profiles is generated using a cancerous sample.

Clause 20. The method of any one of clauses 1-19 wherein the disease is cancer.

Clause 21. The method of claim 20, wherein the cancer is breast cancer or colon cancer.

Clause 22. The method of any one of clauses 20-21, wherein the status is a TNM stage. Clause 23. The method of any one of clauses 1-22, further comprising treating the subject for the disease.

Clause 24. The method of any one of clauses 1 to 23, wherein at least one of the one or more reference spectroscopic profiles is from one or more individuals in the same population as the subject.

Clause 25. The method of clause 24, wherein all the reference spectroscopic profiles are from one or more individuals in the same population as the subject.

Clause 26. The method of any one of clauses 1 to 23, wherein at least one of the one or more reference spectroscopic profiles is from one or more individuals in a different population than the subject.

Clause 27. The method of clause 26, wherein all the reference spectroscopic profiles are from one or more individuals in a different population than the subject.

Clause 28. The method of any one of clauses 1-27, wherein the one or more reference spectroscopic profiles is from the subject.

Clause 29. The method of any one of clauses 1 to 28, wherein the subject is a human.

Clause 30. The method of any one of clauses 1 to 29, wherein the sample is in vitro.

Clause 31. The method of any one of clauses 1 to 30, wherein the sample comprises blood, spittle/saliva, serum, plasma, urine, sputum, sweat, semen, synovial fluids, lymphatic fluids, cerebrospinal fluids, biopsy, stool, or combinations thereof.

Clause 32. The method of any one of clauses 1 to 31, wherein the subject is asymptomatic of the disease.

Clause 33. The method of any one of clauses 1 to 32, wherein the subject is presenting symptoms of the disease.

Clause 34. The method of any one of clauses 1 to 33, wherein the subject has not had or has a prior history of having cancer.

Clause 35. The method of any one of clauses 1 to 34, wherein the subject exhibits one or more symptoms selected from the group consisting of breast pains, breast nodules, nipple discharge, weight loss, fatigue, anemia, and a combination thereof.

Clause 36. The method of any one of clauses 1 to 35, wherein the subject is at risk of developing breast cancer.

Clause 37. The method of any one of clauses 1 to 36, wherein the subject is exposed to one or more assays for identification of breast cancer.

Clause 38. A method for screening for and/or diagnosing a disease in a subject, the method comprising:
(i) generating a spectroscopic profile of the subject's sample, wherein the spectroscopic profile comprises components,
(ii) obtaining a general score of the spectroscopic profile using a computer-implemented technique, and
(iii) providing a diagnosis, prognosis, or both, of the disease based on the general score.

Clause 39. The method of clause 38, wherein the diagnosis comprises comparing the general score to a threshold value, wherein the subject is diagnosed as having the disease when the general score is greater than the threshold.

Clause 40. The method of clause 38 or 39, wherein obtaining the general score comprises using the computer-implemented technique to generate one or more component scores by comparing the components of the spectroscopic profile with corresponding components in at least one of the one or more reference spectroscopic profiles.

Clause 41. The method of clause 40, wherein the general score is obtained by summing the one or more component scores optionally using the computer-implemented technique, wherein when only one component score is available, the general score is that component score.

Clause 42. The method of any one of clauses 38 to 41, wherein the spectroscopic profile of the subject's sample, and the one or more reference profiles are generated using data from a spectroscopic technique that applies a frequency scan between, but not limited to, about 14,000 cm⁻¹ and about 800 cm⁻¹, and sub-ranges within it.

Clause 43. The method of any one of clauses 38 to 42, wherein the spectroscopic profile of the subject's sample, and the one or more reference profiles are generated using data from a spectroscopic technique that applies a frequency scan between, but not limited to, about 14,000 cm⁻¹ and about 800 cm⁻¹, and sub-ranges within it.

Clause 44. The method of any one of clauses 38 to 43, wherein the spectroscopic profile of the subject's sample, and the one or more reference profiles are generated using data from a spectroscopic technique comprising field-resolved spectroscopy (such as field-resolved infrared spectroscopy), frequency-resolved spectroscopy, Fourier-transform infrared spectroscopy, Raman spectroscopy, infrared attenuated total reflectance, diffuse reflectance spectroscopy, and combinations thereof.

Clause 45. The method of clause 44, wherein the spectroscopic technique comprises vibrational spectroscopy.

Clause 46. The method of clause 45, wherein the vibrational spectroscopy comprises infrared spectroscopy, such as near-infrared spectroscopy, mid-infrared, resonant frequency, and/or far-infrared.

Clause 47. The method of any one of clauses 38 to 46, wherein the components of the spectroscopic profile of the subject's sample contain vibrational frequencies.

Clause 48. The method of any one of cl clauses 41 to 47, wherein the components of at least one of the one or more reference spectroscopic profiles contain vibrational frequencies.

Clause 49. The method of any one of clauses 41 to 48, wherein at least one of the one or more reference spectroscopic profiles are generated using a sample from a non-diseased patient.

Clause 50. The method of any one of clauses 41 to 49, wherein at least one of the one or more reference spectroscopic profiles are generated using a sample from a diseased patient.

Clause 51. The method of any one of clauses 41 to 49, wherein at least one of the one or more reference spectroscopic profiles are generated using a cancerous sample.

Clause 52. The method of clause 51, wherein the cancerous sample has cancer selected from the group consisting of BC, lung cancer, prostate cancer, colon cancer, skin cancer, blood cancer (such as leukemia and/or lymphoma), myeloma, and a combination thereof.

Clause 53. The method of any one of clauses 41 to 52, wherein at least one of the one or more reference profiles is from one or more individuals in the same population as the subject.

Clause 54. The method of any one of clauses 41 to 51, wherein all the reference spectroscopic profiles are from one or more individuals in the same population as the subject.

Clause 55. The method of any one of clauses 41 to 50, wherein at least one of the one or more reference spectroscopic profiles is from one or more individuals in a different population than the subject.

Clause 56. The method of any one of clauses 41 to 52, or 55, wherein all the reference spectroscopic profiles are from one or more individuals in a different population than the subject.

Clause 57. The method of any one of clauses 41 to 56, wherein the general score is obtained by comparing the spectroscopic profile with the first reference spectroscopic profile and the second reference spectroscopic profile.

Clause 58. The method of clause 57, wherein the first reference spectroscopic profile contains upper bounds of spectroscopic data.

Clause 59. The method of clause 57 or 58, wherein the second reference spectroscopic profile contains lower bounds of spectroscopic data.

Clause 60. The method of any one of clauses 38 to 59, wherein the subject is a human or other animals.

Clause 61. The method of any one of clauses 38 to 60, wherein the sample is in vitro.

Clause 62. The method of any one of clauses 38 to 61, wherein the disease comprises BC.

Clause 63. The method of any one of clauses 38 to 62, wherein the sample is selected from the group consisting of cells, blood, spittle/saliva, serum, plasma, urine, sputum, sweat, semen, synovial fluids, lymphatic fluids, cerebrospinal fluids, biopsy, stool, and combinations thereof.

Clause 64. The method of any one of clauses 38 to 63, wherein the subject is asymptomatic of the disease.

Clause 65. The method of any one of clauses 38 to 63, wherein the diagnosis is performed on the subject presenting symptoms of the disease.

Clause 66. The method of any one of clauses 38 to 65, wherein the subject has not had or has a prior history of having cancer.

Clause 67. The method of any one of clauses 38 to 64 or 66, wherein the subject exhibits one or more symptoms selected from the group consisting of breast pains, breast nodules, nipple discharge, weight loss, fatigue, anemia, or a combination thereof.

Clause 68. The method of any one of clauses 38 to 67, wherein the subject is at risk (such as at high risk) of developing BC.

Clause 69. The method of any one of clauses 38 to 68, wherein the subject is exposed to one or more assays for identification of BC.

Clause 70. The method of any one of clauses 38 to 69, wherein the general score is used to determine whether the subject has BC.

Clause 71. The method of any one of clauses 38 to 70, wherein the components are ordered by wavenumbers.

Numerous specific details are set forth to provide a thorough understanding of the subject matter described herein. However, it will be understood by those of ordinary skill in the art that the subject matter described herein may be practiced without these specific details. In other instances, well-known methods, procedures, and components have not been described in detail so as not to obscure the subject matter described herein. No example embodiment described limits any claim and any claim may cover processes or systems that differ from those described. The claims are not limited to systems or processes having all the features of any one system or process described above or to features common to multiple or all the systems or processes described above. It is possible that a system or process described above is not an embodiment of any exclusive right granted by issuance of this patent application. Any subject matter described above and for which an exclusive right is not granted by issuance of this patent application may be the subject matter of another protective instrument, for example, a continuing patent application, and the applicants, inventors or owners do not intend to abandon, disclaim or dedicate to the public any such subject matter by its disclosure in this document.

### EXAMPLES

### Example 1: Non-Invasive Diagnostic Test for Breast Cancer Detection Using Raman Spectroscopy

The value of this approach in cancer screening was obtained in the context of BC following the Raman spectroscopy analysis of a series of liquid biopsies from 22 healthy controls (absence of BC by standard mammographic screening) and 24 patients with Stage I and II BC (King Saud University, Riyadh, Saudi Arabia). The Raman analysis was performed at the King Abdullah University of Science and Technology, (KAUST) in Thuwal, Saudi Arabia. A non-hierarchical data mining correlative analysis was performed to classify spectroscopic profiles.

Several existing technologies, allow for high-throughput spectroscopy measurements of biomolecules at low concentration, opening an avenue for cancer detection at an early stage and therapy monitoring.

### Materials and Methods

In a collaboration between the Computational Bioscience Research Group at the King Abdullah University of Science and Technology and the Oncology Center at the King Saud University Medical City, a study was performed on a set of 24 BC and 22 normal control samples.

### Experimental Analysis

Experimental analysis of each sample was performed through Raman spectroscopy using a 10× dry objective and laser wavelength of 785 nm, with circular polarization and intensity of 50 mW at the sample. The spectra were collected using a 600 gr/mm diffraction grating in the Raman spectrometer.

Briefly, blood samples that were collected were prepared, and were analyzed using Raman spectroscopy according to the following steps: Samples were collected in tubes provided for this purpose up to 18.6 mL/sample per patient (tube EDTA K3). The samples were incubated at room temperature until the blood coagulated. The tubes were then centrifuged for 10 minutes at 7000 rpm at room temperature and the supernatants were then aliquoted into Eppendorf tubes (1 mL each serum) and stored at −80° C. No freeze-defreeze cycle was performed. Three aliquots of each serum sample at each sampling time (total 3 mL) were kept for the possibility of carrying out several independent measurements. Tubes were then shipped to KAUST by batches, under cryopreservation with temperature-controlled processes and assessed at KAUST. At KAUST the samples were stored in Eppendorf tubes at −80° C. They were moved at −20° C. for 2 minutes and then into ice for ~3 hours until they were thawed. The Raman micro-spectrometer was calibrated every day twice per day with the reference sample (Silicon sample in our case). A volume of 40 μl of plasma was taken from the Eppendorf tube and placed on a glass microscope slide (Objekttäger, Micro Slides ground 90°, 1 mm thickness) previously covered with Aluminum foil. With the configuration described above, 2 spectra per plasma drop were taken, using an integration time of 30 seconds and 20 accumulations, over a spectral range from 0 cm⁻¹ to 2300 cm⁻¹. The 2 spectral measurements were performed on the other 2 drops, for a total of 3 drops (40 μl each) per patient's sample. After the spectral acquisition, a smoothing of the raw data with the Savitzky-Golay algorithm (polynomial of order 5 ad window 13) was performed. The baseline was reduced by subtracting a water spectrum obtained from the average of 6 water spectra, taken in the same conditions of the blood plasma (40 μl on a microscope slide covered with Aluminum foil). The removal of the baseline was performed using free-ware Raman Tool Set software.

TABLE 1
KSU Study Breast Cancer Clinical Data From Raman Spectroscopy Analysis

| NUM | GROUP | WEIGHT | HEIGHT | BMI | AGE | AGECLASS | AGEPER1 | AGECHIL1 |
|---|---|---|---|---|---|---|---|---|
| 70 | CASE | 71 | 142 | 35.2 | 56 | ≥50 | ≥14 | <30 |
| 71 | CASE | 74 | 157 | 30.0 | 56 | ≥50 | ≥14 | <30 |
| 72 | CASE | 63.4 | 169 | 22.2 | 52 | ≥50 | ≥14 | <30 |
| 73 | CASE | 78 | 158 | 31.2 | 60 | ≥50 | <12 | ≥30 |
| 74 | CASE | 41 | 163 | 15.4 | 46 | <50 | ≥14 | No Birth |
| 75 | CASE | 46.5 | 149 | 20.9 | 67 | ≥50 | ≥14 | <30 |
| 76 | CASE | 57.9 | 160 | 22.6 | 44 | <50 | ≥14 | No Birth |
| 77 | CASE | 78.5 | 147 | 36.3 | 55 | ≥50 | ≥14 | <30 |
| 78 | CONTROL | 94 | 160 | 36.7 | 46 | <50 | ≥12-<14 | <30 |
| 79 | CASE | 73 | 145 | 34.7 | 57 | ≥50 | <12 | <30 |
| 80 | CASE | 85.5 | 154.5 | 35.8 | 57 | ≥50 | <12 | <30 |
| 81 | CASE | 65 | 147 | 30.1 | 55 | ≥50 | <12 | No Birth |
| 82 | CONTROL | 73 | 157 | 29.6 | 36 | <50 | ≥12-<14 | <30 |
| 83 | CONTROL | 55 | 164 | 20.4 | 34 | <50 | ≥12-<14 | No Birth |
| 84 | CASE | 47 | 153 | 20.1 | 70 | ≥50 | ≥12-<14 | <30 |
| 85 | CASE | 67 | 157 | 27.2 | 52 | ≥50 | ≥12-<14 | <30 |
| 86 | CASE | 65 | 155 | 27.1 | 46 | <50 | <12 | No Birth |
| 87 | CASE | 73 | 159 | 28.9 | 35 | <50 | ≥14 | <30 |
| 89 | CONTROL | 53 | 148 | 24.2 | 29 | <50 | ≥14 | <30 |
| 90 | CASE | 95 | 172 | 32.1 | 35 | <50 | ≥12-<14 | No Birth |
| 91 | CASE | 84 | 154 | 35.4 | 60 | ≥50 | ≥14 | <30 |
| 92 | CONTROL | 55 | 158 | 22.0 | 35 | <50 | ≥14 | ≥30 |
| 93 | CONTROL | 69 | 160 | 27.0 | 25 | <50 | ≥12-<14 | No Birth |
| 94 | CONTROL | 75 | 161 | 28.9 | 50 | ≥50 | ≥12-<14 | ≥30 |
| 95 | CONTROL | 115 | 164 | 42.8 | 50 | ≥50 | ≥12-<14 | ≥30 |
| 96 | CONTROL | 74 | 164 | 27.5 | 39 | <50 | <12 | <30 |
| 97 | CONTROL | 75 | 165 | 27.5 | 40 | <50 | ≥12-<14 | No Birth |
| 98 | CONTROL | 68 | 164 | 25.3 | 48 | <50 | ≥14 | No Birth |
| 99 | CONTROL | 80 | 158 | 32.0 | 47 | <50 | <12 | <30 |
| 100 | CONTROL | 62 | 156 | 25.5 | 43 | <50 | <12 | No Birth |
| 101 | CONTROL | 85 | 169 | 29.8 | 24 | <50 | <12 | No Birth |
| 104 | CONTROL | 56 | 156 | 23.0 | 32 | <50 | ≥14 | No Birth |
| 105 | CASE | 51 | 155 | 21.2 | 40 | <50 | ≥14 | No Birth |
| 106 | CASE | 61 | 152 | 26.4 | 57 | ≥50 | ≥12-<14 | <30 |
| 107 | CASE | 73 | 157 | 29.6 | 53 | ≥50 | ≥12-<14 | <30 |
| 108 | CASE | 57 | 150 | 25.3 | 50 | ≥50 | ≥12-<14 | <30 |
| 109 | CONTROL | 80 | 160 | 31.3 | 24 | <50 | ≥14 | No Birth |
| 110 | CASE | 90 | 155 | 37.5 | 62 | ≥50 | ≥14 | <30 |
| 111 | CASE | 105 | 162 | 40.0 | 30 | <50 | ≥12-<14 | No Birth |
| 112 | CONTROL | 71 | 166 | 25.8 | 36 | <50 | ≥12-<14 | No Birth |
| 113 | CONTROL | 84 | 168 | 29.8 | 34 | <50 | ≥12-<14 | ≥30 |
| 114 | CONTROL | 70 | 167 | 25.1 | 31 | <50 | ≥14 | <30 |
| 115 | CONTROL | 56 | 159 | 22.2 | 33 | <50 | ≥12-<14 | No Birth |
| 116 | CONTROL | 59 | 164 | 21.9 | 40 | <50 | ≥12-<14 | <30 |
| 117 | CONTROL | 68 | 163 | 25.6 | 45 | <50 | ≥14 | <30 |
| 118 | CONTROL | 59 | 158 | 23.6 | 29 | <50 | ≥12-<14 | No Birth |
| 119 | CONTROL | 70 | 159 | 27.7 | 48 | <50 | ≥14 | ≥30 |
| 120 | CONTROL | 60 | 170 | 20.8 | 36 | <50 | ≥14 | <30 |
| 121 | CONTROL | 64 | 165 | 23.5 | 61 | ≥50 | ≥14 | ≥30 |
| 122 | CONTROL | 56 | 161 | 21.6 | 31 | <50 | <12 | No Birth |
| 123 | CONTROL | 68 | 161 | 26.2 | 42 | <50 | <12 | ≥30 |
| 124 | CONTROL | | 166 | #VALUE! | 31 | <50 | ≥14 | <30 |
| 125 | CONTROL | 77 | 164 | 28.6 | 32 | <50 | ≥12-<14 | ≥30 |
| 126 | CONTROL | 93 | 163 | 35.0 | 49 | <50 | ≥12-<14 | <30 |
| 127 | CASE | 61 | 160 | 23.8 | 36 | <50 | ≥12-<14 | No Birth |
| 128 | CONTROL | 65 | 156 | 26.7 | 45 | <50 | ≥12-<14 | <30 |
| 129 | CONTROL | 57 | 157 | 23.1 | 33 | <50 | ≥12-<14 | No Birth |
| 130 | CONTROL | 68 | 162 | 25.9 | 35 | <50 | ≥14 | <30 |
| 131 | CASE | 56 | 160 | 21.9 | 41 | <50 | <12 | ≥30 |
| 132 | CONTROL | 56 | 158 | 22.4 | 60 | ≥50 | ≥14 | <30 |
| 133 | CASE | 66 | 170 | 22.8 | 52 | ≥50 | ≥14 | <30 |
| 134 | CONTROL | 52 | 162 | 19.8 | 46 | <50 | ≥12-<14 | <30 |
| 135 | CONTROL | 50 | 164 | 18.6 | 30 | <50 | ≥14 | <30 |
| 136 | CASE | 58 | 142 | 28.8 | 34 | <50 | ≥12-<14 | No Birth |
| 137 | CONTROL | 55 | 153 | 23.5 | 30 | <50 | ≥14 | No Birth |
| 138 | CONTROL | 53 | 148 | 24.2 | 40 | <50 | ≥12-<14 | ≥30 |
| 139 | CONTROL | 86 | 158 | 34.4 | 28 | <50 | ≥14 | No Birth |
| 140 | CONTROL | 63 | 167 | 22.6 | 40 | <50 | ≥12-<14 | <30 |
| 141 | CASE | 82 | 165 | 30.1 | 45 | <50 | <12 | <30 |
| 142 | CONTROL | 52 | 164 | 19.3 | 53 | ≥50 | ≥12-<14 | No Birth |
| 143 | CONTROL | 95 | 162 | 36.2 | 28 | <50 | ≥12-<14 | No Birth |
| 144 | CONTROL | 52 | 156 | 21.4 | 22 | <50 | ≥12-<14 | No Birth |
| 145 | CONTROL | 84 | 163 | 31.6 | 17 | <50 | ≥14 | No Birth |
| 146 | CONTROL | 47 | 158 | 18.8 | 15 | <50 | ≥14 | No Birth |
| 147 | CONTROL | 60 | 156 | 24.7 | 29 | <50 | ≥12-<14 | <30 |
| 148 | CONTROL | 58 | 161 | 22.4 | 28 | <50 | ≥14 | No Birth |
| 149 | CONTROL | 77 | 163 | 29.0 | 32 | <50 | ≥12-<14 | No Birth |
| 150 | CONTROL | 50 | 150 | 22.2 | 28 | <50 | ≥12-<14 | No Birth |
| 151 | CONTROL | 58 | 150 | 25.8 | 31 | <50 | ≥12-<14 | No Birth |
| 152 | CONTROL | 55 | 156 | 22.6 | 33 | <50 | ≥12-<14 | ≥30 |
| 153 | CONTROL | 54 | 156 | 22.2 | 31 | <50 | ≥12-<14 | <30 |
| 154 | CONTROL | 59 | 163 | 22.2 | 28 | <50 | ≥14 | No Birth |
| 155 | CONTROL | 48 | 150 | 21.3 | 30 | <50 | <12 | No Birth |
| 156 | CONTROL | 80 | 172 | 27.0 | 29 | <50 | ≥12-<14 | No Birth |
| 157 | CONTROL | 97 | 164 | 36.1 | 28 | <50 | ≥14 | No Birth |
| 158 | CONTROL | 56 | 162 | 21.3 | 18 | <50 | ≥12-<14 | No Birth |
| 159 | CONTROL | 85 | 182 | 25.7 | 45 | <50 | ≥14 | No Birth |
| 160 | CONTROL | 27 | 160 | 10.5 | 45 | <50 | ≥14 | <30 |
| 161 | CONTROL | 53 | 154 | 22.3 | 29 | <50 | ≥14 | No Birth |
| 162 | CONTROL | 70 | 152 | 30.3 | 45 | <50 | ≥14 | <30 |
| 163 | CONTROL | 52 | 157 | 21.1 | 46 | <50 | ≥12-<14 | <30 |
| 164 | CONTROL | 52 | 152 | 22.5 | 23 | <50 | ≥12-<14 | No Birth |
| 165 | CONTROL | 65 | 164 | 24.2 | 48 | <50 | ≥14 | <30 |
| 166 | CONTROL | 53 | 155 | 22.1 | 39 | <50 | ≥14 | <30 |

| NUM | FAM_BC | MAMM_HD | P_BBIOP | B_ATYHYP | BIO_LCIS | HRAFMENO | PTHORADIO |
|---|---|---|---|---|---|---|---|
| 70 | 0 | 9999 | 9999 | 9999 | 9999 | No | 9999 |
| 71 | 1 | 9999 | 9999 | 9999 | 9999 | N/A | 9999 |
| 72 | 1 | 9999 | 9999 | 9999 | 9999 | No | No |
| 73 | 1 | 9999 | 9999 | 9999 | 9999 | N/A | 9999 |
| 74 | 0 | 9999 | 9999 | 9999 | 9999 | No | 9999 |
| 75 | 0 | 9999 | 9999 | 9999 | 9999 | No | 9999 |
| 76 | 1 | 9999 | 9999 | 9999 | 9999 | No | 9999 |
| 77 | 0 | 9999 | 9999 | 9999 | 9999 | N/A | 9999 |
| 78 | 0 | No | No | No | No | N/A | No |
| 79 | 0 | 9999 | 9999 | 9999 | 9999 | No | 9999 |
| 80 | 2 | 9999 | 9999 | 9999 | 9999 | No | 9999 |
| 81 | 0 | 9999 | 9999 | 9999 | 9999 | No | 9999 |
| 82 | 0 | No | No | No | No | No | No |
| 83 | 0 | No | No | No | No | No | No |
| 84 | 0 | 9999 | 9999 | 9999 | 9999 | No | 9999 |
| 85 | 0 | 9999 | 9999 | 9999 | 9999 | No | 9999 |
| 86 | 1 | 9999 | 9999 | 9999 | 9999 | No | 9999 |
| 87 | 0 | 9999 | 9999 | 9999 | 9999 | No | 9999 |
| 89 | 1 | No | No | No | No | N/A | No |
| 90 | 0 | 9999 | 9999 | 9999 | 9999 | N/A | 9999 |
| 91 | 0 | 9999 | 9999 | 9999 | 9999 | No | 9999 |
| 92 | 0 | No | Yes 1 | No | No | N/A | No |
| 93 | 0 | No | No | No | No | N/A | No |
| 94 | 0 | No | No | No | No | N/A | No |
| 95 | 0 | No | No | No | No | N/A | No |
| 96 | 0 | No | Yes 2 | No | No | N/A | No |
| 97 | 0 | No | No | No | No | N/A | No |
| 98 | 0 | No | No | No | No | N/A | No |
| 99 | 0 | No | No | No | No | N/A | No |
| 100 | 0 | No | No | No | No | N/A | No |
| 101 | 0 | No | No | No | No | N/A | No |
| 104 | 0 | No | No | No | No | N/A | No |
| 105 | 0 | 9999 | 9999 | 9999 | 9999 | No | 9999 |
| 106 | 0 | 9999 | 9999 | 9999 | 9999 | No | 9999 |
| 107 | 1 | 9999 | 9999 | 9999 | 9999 | No | 9999 |
| 108 | 0 | 9999 | 9999 | 9999 | 9999 | No | 9999 |
| 109 | 2 | Yes | No | No | No | N/A | No |
| 110 | 2 | 9999 | 9999 | 9999 | 9999 | N/A | 9999 |
| 111 | 1 | No | Yes 1 | No | No | N/A | Yes |
| 112 | 0 | No | No | No | No | N/A | No |
| 113 | 0 | No | No | No | No | N/A | No |
| 114 | 0 | No | No | No | No | N/A | No |
| 115 | 0 | No | No | No | No | N/A | No |
| 116 | 0 | No | No | No | No | N/A | No |
| 117 | 0 | No | Yes 1 | No | No | N/A | No |
| 118 | 0 | No | No | No | No | N/A | No |
| 119 | 1 | No | No | No | No | N/A | No |
| 120 | 0 | No | No | No | No | N/A | No |
| 121 | 0 | No | No | No | No | N/A | No |
| 122 | 0 | No | No | No | No | N/A | No |
| 123 | 0 | No | Yes 1 | No | No | N/A | No |
| 124 | 0 | No | No | No | No | N/A | No |
| 125 | 0 | No | No | No | No | N/A | No |
| 126 | 0 | No | No | No | No | N/A | No |
| 127 | 0 | 9999 | 9999 | 9999 | 9999 | N/A | 9999 |
| 128 | 0 | No | No | No | No | N/A | No |
| 129 | 0 | No | No | No | No | N/A | No |
| 130 | 0 | No | No | No | No | N/A | No |
| 131 | 0 | 9999 | 9999 | 9999 | 9999 | N/A | 9999 |
| 132 | 0 | No | No | No | No | N/A | No |
| 133 | 1 | 9999 | 9999 | 9999 | 9999 | N/A | 9999 |
| 134 | 0 | No | Yes 1 | No | No | N/A | No |
| 135 | 0 | No | No | No | No | N/A | No |
| 136 | 1 | 9999 | 9999 | 9999 | 9999 | N/A | No |
| 137 | 0 | No | No | No | No | N/A | No |
| 138 | 0 | No | No | No | No | N/A | No |
| 139 | 0 | No | No | No | No | N/A | No |
| 140 | 0 | No | No | No | No | N/A | No |
| 141 | 0 | 9999 | 9999 | 9999 | 9999 | N/A | 9999 |
| 142 | 0 | No | Yes 1 | No | No | N/A | No |
| 143 | 0 | No | No | No | No | N/A | No |
| 144 | 0 | No | No | No | No | N/A | No |
| 145 | 0 | No | No | No | No | N/A | No |
| 146 | 0 | No | No | No | No | N/A | No |
| 147 | 0 | No | No | No | No | N/A | No |
| 148 | 0 | No | No | No | No | N/A | No |
| 149 | 0 | No | No | No | No | N/A | No |
| 150 | 0 | No | No | No | No | N/A | No |
| 151 | 0 | No | No | No | No | N/A | No |
| 152 | 0 | No | No | No | No | N/A | No |
| 153 | 0 | No | No | No | No | N/A | No |
| 154 | 0 | No | No | No | No | N/A | No |
| 155 | 0 | No | Yes 1 | No | No | N/A | No |
| 156 | 0 | No | No | No | No | N/A | No |
| 157 | 0 | No | No | No | No | N/A | No |
| 158 | 0 | No | No | No | No | N/A | No |
| 159 | 0 | No | No | No | No | N/A | No |
| 160 | 0 | No | No | No | No | N/A | No |
| 161 | 0 | No | No | No | No | N/A | No |
| 162 | 0 | No | No | No | No | N/A | No |
| 163 | 0 | No | No | No | No | N/A | No |
| 164 | 0 | No | No | No | No | N/A | No |
| 165 | 0 | No | No | No | No | N/A | No |
| 166 | 0 | 9999 | 9999 | 9999 | 9999 | N/A | 9999 |

| NUM | BRCA1_2 | FBRCA1_2 | PRIORHBC | HISTPATD | HISTO_TY | TGRADE_S | TDIFFE_S |
|---|---|---|---|---|---|---|---|
| 70 | No | No | Yes | Sep-12 | Invasive Ductal carcinoma | Grade 3 | 8 |
| 71 | No | No | Yes | 1 Oct. 2017 | Invasive Ductal carcinoma | Grade 3 | 8 |
| 72 | No | No | Yes | 29 Oct. 2017 | Invasive Ductal carcinoma | Grade 3 | 8 |
| 73 | No | No | Yes | 11 May 2017 | Invasive Ductal carcinoma | Grade 3 | 8 |
| 74 | No | No | Yes | 2017 Jul. 12 | Invasive Ductal carcinoma | Grade 3 | 8 |
| 75 | No | 9999 | Yes | 2017 Jun. 12 | Metaplastic | Grade 3 | 8 |
| 76 | No | No | Yes | 2017 Jul. 31 | Invasive Ductal carcinoma | Grade 2 | 8 |
| 77 | No | No | Yes | 2016 May 8 | Invasive Ductal carcinoma | Grade 3 | 8 |
| 78 | No | No | No | N/A | | | |
| 79 | No | No | Yes | 2017 Apr. 25 | Invasive Ductal carcinoma | Grade 3 | 8 |
| 80 | No | No | Yes | 2016 May 5 | Invasive Ductal carcinoma | Grade 2 | 6 |
| 81 | No | No | Yes | 2005 Feb. 22 | Mucinous (colloid) | 9999 | 9999 |
| 82 | No | No | No | N/A | | | |
| 83 | No | No | No | N/A | | | |
| 84 | No | No | Yes | 2017 Apr. 9 | Invasive Ductal carcinoma | Grade 3 | 9 |
| 85 | No | No | Yes | 2017 Mar. 13 | Invasive Ductal carcinoma | Grade 3 | 8 |
| 86 | No | No | Yes | 2017 Apr. 10 | Invasive Ductal carcinoma | Grade 2 | 6 |
| 87 | No | No | Yes | 2017 Sep. 19 | Invasive Ductal carcinoma | Grade 3 | 9 |
| 89 | No | No | No | N/A | | | |
| 90 | No | No | Yes | 2017 Jun. 18 | Invasive Ductal carcinoma | Grade 3 | 8 |
| 91 | No | No | Yes | 2017 Apr. 13 | Invasive Ductal carcinoma | Grade 3 | 8 |
| 92 | No | No | No | N/A | | | |
| 93 | No | No | No | N/A | | | |
| 94 | No | No | No | N/A | | | |
| 95 | No | No | No | N/A | | | |
| 96 | No | No | No | N/A | | | |
| 97 | No | No | No | N/A | | | |
| 98 | No | No | No | N/A | | | |
| 99 | No | No | No | N/A | | | |
| 100 | No | No | No | N/A | | | |
| 101 | No | No | No | N/A | | | |
| 104 | No | No | No | N/A | | | |
| 105 | No | No | Yes | 2017 Jul. 16 | Invasive Ductal carcinoma | Grade 3 | 8 |
| 106 | No | No | Yes | 2016 Dec. 15 | Invasive Ductal carcinoma | Grade 3 | 9 |
| 107 | No | No | Yes | 2014 Jul. 24 | Invasive Ductal carcinoma | Grade 3 | 9 |
| 108 | No | No | Yes | Jul-14 | mammary carcinoma | Grade 3 | 9999 |
| 109 | No | No | No | N/A | | | |
| 110 | No | No | Yes | 2018 Oct. 16 | Invasive Ductal carcinoma | Grade 3 | 7 |
| 111 | No | No | Yes | 2018 Oct. 3 | Invasive Ductal carcinoma | Grade 3 | 9999 |
| 112 | No | No | No | N/A | | | |
| 113 | No | No | No | N/A | | | |
| 114 | No | No | No | N/A | | | |
| 115 | No | No | No | N/A | | | |
| 116 | No | No | No | N/A | | | |
| 117 | No | No | No | N/A | | | |
| 118 | No | No | No | N/A | | | |
| 119 | No | No | No | N/A | | | |
| 120 | No | No | No | N/A | | | |
| 121 | No | No | No | N/A | | | |
| 122 | No | No | No | N/A | | | |
| 123 | No | No | No | N/A | | | |
| 124 | No | No | No | N/A | | | |
| 125 | No | No | No | N/A | | | |
| 126 | No | No | No | N/A | | | |
| 127 | No | No | Yes | 2015 Jun. 21 | Invasive Ductal carcinoma | Grade 3 | 8 |
| 128 | No | No | No | N/A | | | |
| 129 | No | No | No | N/A | | | |
| 130 | No | No | No | N/A | | | |
| 131 | No | No | Yes | 2018 Nov. 22 | Invasive Ductal carcinoma | Grade 3 | 9 |
| 132 | No | No | No | N/A | | | |
| 133 | No | No | Yes | 2017 Jul. 5 | Invasive Ductal carcinoma | Grade | 8 |
| 134 | No | No | No | N/A | | | |
| 135 | No | No | No | N/A | | | |
| 136 | No | No | Yes | 2017 Dec. 24 | Invasive Ductal carcinoma | Grade 3 | 8 |
| 137 | No | No | No | N/A | | | |
| 138 | No | No | No | N/A | | | |
| 139 | No | No | No | N/A | | | |
| 140 | No | No | No | N/A | | | |
| 141 | No | No | Yes | 2015 Jan. 19 | Invasive Ductal carcinoma | 9999 | 9999 |
| 142 | No | No | No | N/A | | | |
| 143 | No | No | No | N/A | | | |
| 144 | No | No | No | N/A | | | |
| 145 | No | No | No | N/A | | | |
| 146 | No | No | No | N/A | | | |
| 147 | No | No | No | N/A | | | |
| 148 | No | No | No | N/A | | | |
| 149 | No | No | No | N/A | | | |
| 150 | No | No | No | N/A | | | |
| 151 | No | No | No | N/A | | | |
| 152 | No | No | No | N/A | | | |
| 153 | No | No | No | N/A | | | |
| 154 | No | No | No | N/A | | | |
| 155 | No | No | No | N/A | | | |
| 156 | No | No | No | N/A | | | |
| 157 | No | No | No | N/A | | | |
| 158 | No | No | No | N/A | | | |
| 159 | No | No | No | N/A | | | |
| 160 | No | No | No | N/A | | | |
| 161 | No | No | No | N/A | | | |
| 162 | No | No | No | N/A | | | |
| 163 | No | No | No | N/A | | | |
| 164 | No | No | No | N/A | | | |
| 165 | No | No | No | N/A | | | |
| 166 | No | No | Yes | | | | |

| NUM | NUC_PLEO | MITOS_S | TUBUL_F | Ki67_PER | Ki67_CLAS | ER | ER_PERC | ER_ALLRED | PR |
|---|---|---|---|---|---|---|---|---|---|
| 70 | 3 | 2 | 3 | 9999 | 9999 | Negative | 9999 | 9999 | Negative |
| 71 | 3 | 3 | 2 | 40 | ≥14 | Negative | 9999 | 9999 | Negative |
| 72 | 3 | 2 | 3 | 60 | ≥14 | Negative | 9999 | 9999 | Negative |
| 73 | 3 | 2 | 3 | 80 | ≥14 | Positive | 70 | 9999 | Negative |
| 74 | 3 | 2 | 3 | 50 | ≥14 | Positive | 80 | 8/8 | Positive |
| 75 | 3 | 3 | 2 | 50 | ≥14 | Positive | 50 | 9999 | Negative |
| 76 | 3 | 2 | 2 | 60 | ≥14 | Positive | 90 | 9999 | Positive |
| 77 | 3 | 2 | 3 | 35 | ≥14 | Positive | 80 | 9999 | Positive |
| 78 | | | | | | | | | |
| 79 | 3 | 2 | 3 | 70 | ≥14 | Positive | 90 | 8/8 | Positive |
| 80 | 2 | 1 | 3 | 40 | ≥14 | Positive | 90 | 8/8 | Positive |
| 81 | 9999 | 9999 | 9999 | 9999 | 9999 | Negative | 9999 | 9999 | Positive |
| 82 | | | | | | | | | |
| 83 | | | | | | | | | |
| 84 | 3 | 3 | 3 | 50 | ≥14 | Negative | 9999 | 9999 | Negative |
| 85 | 3 | 2 | 3 | 60 | ≥14 | Positive | 5 | 4 | Negative |
| 86 | 2 | 1 | 3 | 15 | ≥14 | Positive | 80 | 9999 | Positive |
| 87 | 3 | 3 | 3 | 80 | ≥14 | Positive | 20 | 5 | Negative |
| 89 | | | | | | | | | |
| 90 | 2 | 3 | 3 | 70 | ≥14 | Negative | 9999 | 9999 | Negative |
| 91 | 3 | 2 | 3 | 45 | ≥14 | Negative | 9999 | 9999 | Negative |
| 92 | | | | | | | | | |
| 93 | | | | | | | | | |
| 94 | | | | | | | | | |
| 95 | | | | | | | | | |
| 96 | | | | | | | | | |
| 97 | | | | | | | | | |
| 98 | | | | | | | | | |
| 99 | | | | | | | | | |
| 100 | | | | | | | | | |
| 101 | | | | | | | | | |
| 104 | | | | | | | | | |
| 105 | 2 | 3 | 3 | 70 | ≥14 | Positive | 80 | 8 | Positive |
| 106 | 3 | 3 | 3 | 9999 | 9999 | Positive | 5 | 9999 | Positive |
| 107 | 3 | 3 | 3 | 40 | ≥14 | Positive | <10 | 9999 | Positive |
| 108 | 9999 | 9999 | 9999 | 9999 | 9999 | Positive | 9999 | 9999 | Positive |
| 109 | | | | | | | | | |
| 110 | 2 | 2 | 3 | 25 | ≥14 | Positive | 95 | 9999 | Positive |
| 111 | 9999 | 9999 | 9999 | 9999 | 9999 | Negative | 9999 | 9999 | Negative |
| 112 | | | | | | | | | |
| 113 | | | | | | | | | |
| 114 | | | | | | | | | |
| 115 | | | | | | | | | |
| 116 | | | | | | | | | |
| 117 | | | | | | | | | |
| 118 | | | | | | | | | |
| 119 | | | | | | | | | |
| 120 | | | | | | | | | |
| 121 | | | | | | | | | |
| 122 | | | | | | | | | |
| 123 | | | | | | | | | |
| 124 | | | | | | | | | |
| 125 | | | | | | | | | |
| 126 | | | | | | | | | |
| 127 | 3 | 2 | 3 | 40 | ≥14 | Negative | 9999 | 9999 | Negative |
| 128 | | | | | | | | | |
| 129 | | | | | | | | | |
| 130 | | | | | | | | | |
| 131 | 3 | 3 | 3 | 9999 | 9999 | Negative | 9999 | 9999 | Negative |
| 132 | | | | | | | | | |
| 133 | 3 | 2 | 3 | 60 | ≥14 | Negative | 9999 | 9999 | Negative |
| 134 | | | | | | | | | |
| 135 | | | | | | | | | |
| 136 | 3 | 2 | 3 | 40 | ≥14 | Negative | 9999 | 9999 | Negative |
| 137 | | | | | | | | | |
| 138 | | | | | | | | | |
| 139 | | | | | | | | | |
| 140 | | | | | | | | | |
| 141 | 9999 | 9999 | 9999 | High | ≥14 | Negative | 9999 | 9999 | Negative |
| 142 | | | | | | | | | |
| 143 | | | | | | | | | |
| 144 | | | | | | | | | |
| 145 | | | | | | | | | |
| 146 | | | | | | | | | |
| 147 | | | | | | | | | |
| 148 | | | | | | | | | |
| 149 | | | | | | | | | |
| 150 | | | | | | | | | |
| 151 | | | | | | | | | |
| 152 | | | | | | | | | |
| 153 | | | | | | | | | |
| 154 | | | | | | | | | |
| 155 | | | | | | | | | |
| 156 | | | | | | | | | |
| 157 | | | | | | | | | |
| 158 | | | | | | | | | |
| 159 | | | | | | | | | |
| 160 | | | | | | | | | |
| 161 | | | | | | | | | |
| 162 | | | | | | | | | |
| 163 | | | | | | | | | |
| 164 | | | | | | | | | |
| 165 | | | | | | | | | |
| 166 | | | | | | | | | |

| NUM | PR_PERC | PR ALLRED | Her2_IMH | Her2_FSH | Her2 | MOL_STYPE | pT | pN | pM |
|---|---|---|---|---|---|---|---|---|---|
| 70 | 9999 | 9999 | 3+ | 9999 | Positive | Her 2 Enriched | T2 | N0 | M0 |
| 71 | 9999 | 9999 | 3+ | 9999 | Positive | Her 2 Enriched | T3 | N1 | M1 |
| 72 | 9999 | 9999 | 3+ | 9999 | Positive | Her 2 Enriched | T1c | N1 | M0 |
| 73 | 9999 | 9999 | 3+ | 9999 | Positive | Her 2 luminal | T2 | N0 | M0 |
| 74 | 10 | 4/8 | 1+ | 9999 | Negative | Luminal B | T4 | N2 | M1 |
| 75 | 9999 | 9999 | 0 | 9999 | Negative | Luminal B | T1c | N3a | M0 |
| 76 | 90 | 9999 | 2+ | Negative | Negative | Luminal B | T4c | N3 | M1 |
| 77 | 20 | 9999 | 0 | 9999 | Negative | Luminal B | T4 | pN0(I+) | M1 |
| 78 | | | | | | | | | |
| 79 | 10 | 4/8 | 2+ | Positive | Positive | Her 2 luminal | T3 | No | M0 |
| 80 | 25 | 5/8 | 0 | 9999 | Negative | Luminal B | T1c | pN1c | M0 |
| 81 | 10 | 9999 | 3+ | 9999 | Positive | Her 2 luminal | TX | NX | M1 |
| 82 | | | | | | | | | |
| 83 | | | | | | | | | |
| 84 | 9999 | 9999 | 1+ | 9999 | Negative | Triple negative | T4b | N2a | M1 |
| 85 | <1 | 2 | 3+ | 9999 | Positive | Her 2 luminal | T1b | N0 | M0 |
| 86 | 70 | 9999 | 1+ | 9999 | Negative | Luminal A | T1b | N0 | M0 |
| 87 | 9999 | 9999 | 0 | 9999 | Negative | Luminal B | T3 | N0 | M1 |
| 89 | | | | | | | | | |
| 90 | 9999 | 9999 | 3+ | 9999 | Positive | Her 2 Enriched | T2 | pN1mi | M0 |
| 91 | 9999 | 9999 | 3+ | 9999 | Positive | Her 2 Enriched | T1a | pN2 | M0 |
| 92 | | | | | | | | | |
| 93 | | | | | | | | | |
| 94 | | | | | | | | | |
| 95 | | | | | | | | | |
| 96 | | | | | | | | | |
| 97 | | | | | | | | | |
| 98 | | | | | | | | | |
| 99 | | | | | | | | | |
| 100 | | | | | | | | | |
| 101 | | | | | | | | | |
| 104 | | | | | | | | | |
| 105 | 10 | 4 | 2+ | Negative | Negative | Luminal B | T2 | N0 | M0 |
| 106 | 5 | 9999 | 2+ | Positive | Positive | Her 2 luminal | T2 | N0 | M0 |
| 107 | >90 | 9999 | 3+ | 9999 | Positive | Her 2 Enriched | 9999 | 9999 | M1 |
| 108 | 9999 | 9999 | 3+ | 9999 | Positive | Her 2 Enriched | 9999 | 9999 | M1 |
| 109 | | | | | | | | | |
| 110 | 15 | 9999 | 1+ | 9999 | Negative | Luminal B | T3 | N1 | M1 |
| 111 | 9999 | 9999 | 1+ | 9999 | Negative | Triple negative | T2 | N0 | M0 |
| 112 | | | | | | | | | |
| 113 | | | | | | | | | |
| 114 | | | | | | | | | |
| 115 | | | | | | | | | |
| 116 | | | | | | | | | |
| 117 | | | | | | | | | |
| 118 | | | | | | | | | |
| 119 | | | | | | | | | |
| 120 | | | | | | | | | |
| 121 | | | | | | | | | |
| 122 | | | | | | | | | |
| 123 | | | | | | | | | |
| 124 | | | | | | | | | |
| 125 | | | | | | | | | |
| 126 | | | | | | | | | |
| 127 | 9999 | 9999 | 3+ | 9999 | Positive | Her 2 Enriched | T2 | N1 | M0 |
| 128 | | | | | | | | | |
| 129 | | | | | | | | | |
| 130 | | | | | | | | | |
| 131 | 9999 | 9999 | 3+ | 9999 | Positive | Her 2 Enriched | T1c | pN1a | M0 |
| 132 | | | | | | | | | |
| 133 | 9999 | 9999 | 3+ | 9999 | Positive | Her 2 Enriched | T1c | pN1a | M0 |
| 134 | | | | | | | | | |
| 135 | | | | | | | | | |
| 136 | 9999 | 9999 | 3+ | 9999 | Positive | Her 2 Enriched | T3 | N1 | M0 |
| 137 | | | | | | | | | |
| 138 | | | | | | | | | |
| 139 | | | | | | | | | |
| 140 | | | | | | | | | |
| 141 | 9999 | 9999 | 3+ | 9999 | Positive | Her 2 Enriched | TX | NX | M1 |
| 142 | | | | | | | | | |
| 143 | | | | | | | | | |
| 144 | | | | | | | | | |
| 145 | | | | | | | | | |
| 146 | | | | | | | | | |
| 147 | | | | | | | | | |
| 148 | | | | | | | | | |
| 149 | | | | | | | | | |
| 150 | | | | | | | | | |
| 151 | | | | | | | | | |
| 152 | | | | | | | | | |
| 153 | | | | | | | | | |
| 154 | | | | | | | | | |
| 155 | | | | | | | | | |
| 156 | | | | | | | | | |
| 157 | | | | | | | | | |
| 158 | | | | | | | | | |
| 159 | | | | | | | | | |
| 160 | | | | | | | | | |
| 161 | | | | | | | | | |
| 162 | | | | | | | | | |
| 163 | | | | | | | | | |
| 164 | | | | | | | | | |
| 165 | | | | | | | | | |
| 166 | | | | | | | | | |

| NUM | STAGE | RISK_SC | STAGE_GP | NOTE |
|---|---|---|---|---|
| 70 | Stage IIA | | WOMAN WITH BC STAGE I-IIIA | 5 |
| 71 | Stage IV | | WOMAN WITH BC STAGE IIIB-IV | 5 |
| 72 | Stage IIA | | WOMAN WITH BC STAGE I-IIIA | 6 |
| 73 | Stage IIA | | WOMAN WITH BC STAGE I-IIIA | 7 |
| 74 | Stage IV | | WOMAN WITH BC STAGE IIIB-IV | 8 |
| 75 | Stage IIIC | | WOMAN WITH BC STAGE IIIB-IV | 8 |
| 76 | Stage IV | | WOMAN WITH BC STAGE IIIB-IV | 6 |
| 77 | Stage IV | | WOMAN WITH BC STAGE IIIB-IV | 8 |
| 78 | | | | Excluded (7) |
| 79 | Stage IIB | | WOMAN WITH BC STAGE I-IIIA | 7 |
| 80 | Stage IIA | | WOMAN WITH BC STAGE I-IIIA | 7 |
| 81 | Stage IV | | WOMAN WITH BC STAGE IIIB-IV | 7 |
| 82 | | | | 8 |
| 83 | | | | 8 |
| 84 | Stage IV | | WOMAN WITH BC STAGE IIIB-IV | 8 |
| 85 | Stage IA | | WOMAN WITH BC STAGE I-IIIA | 8 |
| 86 | Stage IA | | WOMAN WITH BC STAGE I-IIIA | 8 |
| 87 | Stage IV | | WOMAN WITH BC STAGE IIIB-IV | 8 |
| 89 | | | | Low Risk (8) |
| 90 | Stage IIB | | WOMAN WITH BC STAGE I-IIIA | 8 |
| 91 | Stage IIIA | | WOMAN WITH BC STAGE I-IIIA | 8 |
| 92 | | | | 7 |
| 93 | | | | 7 |
| 94 | | | | 8 |
| 95 | | | | 8 |
| 96 | | | | 8 |
| 97 | | | | 7 |
| 98 | | | | 8 |
| 99 | | | | 8 |
| 100 | | | | 9 |
| 101 | | | | 6 |
| 104 | | | | 8 |
| 105 | Stage IIA | | WOMAN WITH BC STAGE I-IIIA | 8 |
| 106 | Stage IIA | | WOMAN WITH BC STAGE I-IIIA | 8 |
| 107 | Stage IV | | WOMAN WITH BC STAGE IIIB-IV | 8 |
| 108 | Stage IV | | WOMAN WITH BC STAGE IIIB-IV | 8 |
| 109 | | | | 8 |
| 110 | Stage IV | | WOMAN WITH BC STAGE IIIB-IV | 8 |
| 111 | Stage IIA | | WOMAN WITH BC STAGE I-IIIA | 8 |
| 112 | | | | 8 |
| 113 | | | | 8 |
| 114 | | | | |
| 115 | | | | |
| 116 | | | | |
| 117 | | | | |
| 118 | | | | |
| 119 | | | | |
| 120 | | | | |
| 121 | | | | |
| 122 | | | | |
| 123 | | | | |
| 124 | | | | |
| 125 | | | | |
| 126 | | | | |
| 127 | Stage IIB | 9999 | WOMAN WITH BC STAGE I-IIIA | |
| 128 | | | | |
| 129 | | | | |
| 130 | | | | |
| 131 | Stage IIA | 9999 | WOMAN WITH BC STAGE I-IIIA | |
| 132 | | | | |
| 133 | Stage IIA | 9999 | WOMAN WITH BC STAGE I-IIIA | |
| 134 | | | | |
| 135 | | | | |
| 136 | Stage IIIA | 9999 | WOMAN WITH BC STAGE I-IIIA | |
| 137 | | | | |
| 138 | | | | |
| 139 | | | | |
| 140 | | | | |
| 141 | Stage IV | 9999 | WOMAN WITH BC STAGE IIIB-IV | |
| 142 | | | | |
| 143 | | | | |
| 144 | | | | |
| 145 | | | | |
| 146 | | | | |
| 147 | | | | |
| 148 | | | | |
| 149 | | | | |
| 150 | | | | |
| 151 | | | | |
| 152 | | | | |
| 153 | | | | |
| 154 | | | | |
| 155 | | | | |
| 156 | | | | |
| 157 | | | | |
| 158 | | | | |
| 159 | | | | |
| 160 | | | | |
| 161 | | | | |
| 162 | | | | |
| 163 | | | | |
| 164 | | | | |
| 165 | | | | |
| 166 | | | | |

| NUM | COMMENT |
|---|---|
| 70 | Treated before on follow up, MRM in September 2012, |
| | CTX TC *4, Herceptin *18 finished in November 2013 |
| 71 | |
| 72 | |
| 73 | having hx of endometrious cancer diagnosed in 2007 |
| 74 | |
| 75 | |
| 76 | |
| 77 | |
| 78 | |
| 79 | |
| 80 | |
| 81 | |
| 82 | |
| 83 | |
| 84 | |
| 85 | |
| 86 | |
| 87 | to be clarify staging |
| 89 | |
| 90 | |
| 91 | |
| 92 | |
| 93 | |
| 94 | |
| 95 | |
| 96 | |
| 97 | |
| 98 | |
| 99 | |
| 100 | |
| 101 | |
| 104 | |
| 105 | |
| 106 | |
| 107 | |
| 108 | |
| 109 | |
| 110 | |
| 111 | |
| 112 | |
| 113 | |
| 114 | |
| 115 | |
| 116 | |
| 117 | |
| 118 | |
| 119 | |
| 120 | |
| 121 | |
| 122 | |
| 123 | |
| 124 | |
| 125 | |
| 126 | |
| 127 | |
| 128 | |
| 129 | |
| 130 | |
| 131 | |
| 132 | |
| 133 | |
| 134 | |
| 135 | |
| 136 | |
| 137 | |
| 138 | |
| 139 | |
| 140 | |
| 141 | |
| 142 | |
| 143 | |
| 144 | |
| 145 | |
| 146 | |
| 147 | |
| 148 | |
| 149 | |
| 150 | |
| 151 | |
| 152 | |
| 153 | |
| 154 | |
| 155 | |
| 156 | |
| 157 | |
| 158 | |
| 159 | |
| 160 | |
| 161 | |
| 162 | |
| 163 | |
| 164 | |
| 165 | |
| 166 | |

TABLE 2
KSA Colorectal Clinical Data From Raman Spectroscopy (Collected in KSA at Site Number 101-001)

| Number | Cohort | Age | Sex | Weight | Height | BMI |
|---|---|---|---|---|---|---|
| 1 | Patient | 71 | Male | 61.9 | 1.57 | 25.1 |
| 2 | Patient | 65 | Male | 41 | 1.65 | 15.1 |
| 3 | Patient | 59 | Male | 99 | 1.61 | 38.2 |
| 4 | Patient | 53 | Male | 86 | 1.73 | 28.7 |
| 5 | Control | 51 | Female | 50.4 | 1.53 | 21.5 |
| 6 | Control | 65 | Female | 75 | 1.68 | 26.6 |
| 7 | Control | 23 | Female | 52 | 1.59 | 20.6 |
| 8 | Patient | 49 | Female | 66 | 1.56 | 27.1 |
| 9 | Control | 21 | Male | 54.5 | 1.86 | 15.8 |
| 10 | Control | 41 | Male | 122.3 | 1.65 | 44.9 |
| 11 | Control | 26 | Female | 57 | 1.55 | 23.7 |
| 12 | Control | 26 | Male | 49.3 | 1.59 | 19.5 |
| 13 | Control | 47 | Female | 86.9 | 1.46 | 40.8 |
| 14 | Control | 58 | Female | 63.4 | 1.5 | 28.2 |
| 15 | Control | 30 | Male | 68.9 | 1.68 | 24.4 |
| 16 | Control | 32 | Male | 45.7 | 1.65 | 16.8 |
| 17 | Patient | 72 | Male | 52 | 1.63 | 19.6 |
| 18 | Control | 27 | Female | 45.4 | 1.57 | 18.4 |
| 19 | Control | 75 | Male | 82.8 | 1.62 | 31.6 |
| 20 | Control | 66 | Male | 88.7 | 1.76 | 28.6 |
| 21 | Control | 54 | Male | 77.8 | 1.58 | 31.2 |
| 22 | Control | 55 | Male | 73.9 | 1.66 | 26.8 |
| 23 | Control | 24 | Female | 44.7 | 1.7 | 15.5 |
| 24 | Control | 49 | Female | 70.3 | 1.54 | 29.6 |
| 25 | Control | 28 | Male | 54.4 | 1.72 | 18.4 |
| 26 | Control | 21 | Male | 64 | 1.64 | 23.8 |
| 27 | Control | 59 | Male | 73 | 1.73 | 24.4 |
| 28 | Control | 58 | Female | 64 | 1.48 | 29.2 |
| 29 | Control | 20 | Male | 101 | 1.78 | 31.9 |
| 30 | Control | 21 | Male | 67.5 | 1.73 | 22.6 |
| 31 | Control | 47 | Female | 64.4 | 1.55 | 26.8 |
| 32 | Control | 19 | Female | 56.2 | 1.6 | 22 |
| 33 | Control | 62 | Male | 80 | 1.62 | 30.5 |
| 34 | Control | 40 | Male | 79.6 | 1.75 | 26 |
| 35 | Control | 50 | Female | 92.5 | 1.6 | 36.1 |
| 36 | Control | 37 | Female | . | . | . |
| 37 | Patient | 52 | Male | . | . | . |
| 38 | Patient | 52 | Male | . | . | . |
| 39 | Patient | 62 | Male | . | . | . |
| 40 | Patient | 56 | Female | . | . | . |
| 41 | Patient | 74 | Male | . | . | . |
| 42 | Control | 41 | Male | . | . | . |
| 43 | Patient | 68 | Male | . | . | . |
| 44 | Control | 68 | Male | . | . | . |
| 45 | Control | 62 | Female | . | . | . |
| 46 | Control | 31 | Female | . | . | . |
| 47 | Control | 41 | Male | . | . | . |
| 48 | Patient | 54 | Male | . | . | . |
| 49 | Control | 24 | Male | . | . | . |
| 50 | Patient | 38 | Female | . | . | . |
| 51 | Control | 26 | Male | . | . | . |
| 52 | Control | 49 | Female | . | . | . |
| 53 | Control | 71 | Male | 87.8 | 1.63 | 33 |
| 54 | Control | 50 | Female | 68 | 1.56 | 27.9 |
| 55 | Control | 21 | Female | 46 | 1.52 | 19.9 |
| 56 | Control | 29 | Male | 61 | 1.71 | 20.9 |
| 57 | Control | 60 | Male | 63 | 1.57 | 25.6 |
| 58 | Control | 44 | Male | 96 | 1.68 | 34 |
| 59 | Control | 38 | Female | 62 | 1.62 | 23.6 |
| 60 | Control | 51 | Male | 105 | 1.7 | 36.3 |
| 61 | Control | 75 | Female | 81 | 1.5 | 36 |
| 62 | Control | 56 | Male | 112 | 1.68 | 39.7 |
| 63 | Control | 50 | Male | 88 | 1.79 | 27.5 |
| 64 | Control | 60 | Female | 74 | 1.62 | 28.2 |
| 65 | Control | 58 | Male | 73 | 1.73 | 24.4 |
| 66 | Control | 29 | Female | 49 | 1.54 | 20.7 |
| 67 | Control | 67 | Female | 80 | 1.59 | 31.6 |
| 68 | Control | 42 | Female | 82 | 1.56 | 33.7 |
| 69 | Control | 28 | Female | 65 | 1.58 | 26 |
| 70 | Control | 51 | Female | 68 | 1.59 | 26.9 |
| 71 | Control | 29 | Male | 62 | 1.8 | 19.1 |
| 72 | Control | 60 | Male | 71 | 1.71 | 24.3 |
| 73 | Control | 75 | Male | 83 | 1.66 | 30.1 |
| 74 | Control | 36 | Female | 82 | 1.7 | 28.4 |
| 75 | Control | 62 | Female | 75.9 | 1.49 | 34.2 |
| 76 | Control | 34 | Female | 74 | 1.57 | 30 |
| 77 | Control | 19 | Female | 133.8 | 1.7 | 46.3 |
| 78 | Control | 45 | Male | 108 | 1.76 | 34.9 |
| 79 | Control | 57 | Female | 50.2 | 1.43 | 24.5 |
| 80 | Control | 23 | Male | 83.7 | 1.76 | 27 |
| 81 | Control | 27 | Female | 42.8 | 1.44 | 20.6 |
| 82 | Patient | 62 | Male | 124 | 1.7 | 42.9 |
| 83 | Control | 36 | Male | 70.9 | 1.69 | 24.8 |
| 84 | Control | 27 | Female | 57.1 | 1.58 | 22.9 |
| 85 | Control | 42 | Female | 72 | 1.43 | 35.2 |
| 86 | Control | 30 | Male | 85.1 | 1.83 | 25.4 |
| 87 | Control | 25 | Female | 51 | 1.51 | 22.4 |
| 88 | Control | 62 | Male | 101 | 1.64 | 37.6 |
| 89 | Control | 63 | Male | 82 | 1.72 | 27.7 |
| 90 | Control | 62 | Female | 79.7 | 1.55 | 33.2 |
| 91 | Control | 51 | Male | 85.5 | 1.73 | 28.6 |
| 92 | Control | 35 | Male | 69 | 1.75 | 22.5 |
| 93 | Control | 61 | Female | 113 | 1.7 | 39.1 |
| 94 | Control | 61 | Female | 62.7 | 1.51 | 27.5 |
| 95 | Control | 45 | Female | 105.5 | 1.61 | 40.7 |
| 96 | Control | 60 | Female | 48 | 1.58 | 19.2 |
| 97 | Control | 59 | Female | 65 | 1.69 | 22.8 |
| 98 | Control | 63 | Male | 75.3 | 1.6 | 29.4 |
| 99 | Control | 51 | Female | 64.4 | 1.59 | 25.5 |
| 100 | Control | 32 | Male | 61 | 1.77 | 19.5 |
| 101 | Control | 58 | Male | 77 | 1.73 | 25.7 |
| 102 | Control | 64 | Male | 53.3 | 1.55 | 22.2 |
| 103 | Patient | 49 | Male | 71 | 1.7 | 24.6 |
| 104 | Control | 48 | Female | 88 | 1.56 | 36.2 |
| 105 | Control | 21 | Male | 68.5 | 1.69 | 24 |
| 106 | Control | 82 | Male | 70.6 | 1.63 | 26.6 |
| 107 | Control | 36 | Female | 75 | 1.65 | 27.5 |
| 108 | Patient | 71 | Male | 44.7 | 1.47 | 20.7 |
| 109 | Patient | 76 | Male | . | . | . |
| 110 | Control | 36 | Male | 64.8 | 1.7 | 22.4 |
| 111 | Control | 44 | Male | 87.5 | 1.64 | 32.5 |
| 112 | Control | 57 | Male | 70 | 1.68 | 24.8 |
| 113 | Control | 48 | Female | 85 | 1.53 | 36.3 |
| 114 | Patient | 46 | Female | 75 | 1.55 | 31.2 |
| 115 | Patient | 55 | Male | 65.7 | 1.67 | 23.6 |
| 116 | Patient | 47 | Male | 94 | 1.85 | 27.5 |
| 117 | Patient | 74 | Male | 73 | 1.68 | 25.9 |
| 118 | Patient | 63 | Male | 75 | 1.75 | 24.5 |
| 119 | Patient | 47 | Male | 66.5 | 1.65 | 24.4 |
| 120 | Patient | 49 | Female | 67 | 1.6 | 26.2 |
| 121 | Patient | 65 | Male | 67.8 | 1.66 | 24.6 |
| 122 | Patient | 48 | Female | 82.6 | 1.65 | 30.3 |
| 123 | Patient | 62 | Male | 86 | 1.68 | 30.5 |
| 124 | Control | 75 | Male | 77 | 1.7 | 26.6 |
| 125 | Patient | 35 | Male | 70.6 | 1.71 | 24.1 |
| 126 | Control | 63 | Male | 60.8 | 1.76 | 19.6 |
| 127 | Control | 69 | Female | 133 | 1.66 | 48.3 |
| 128 | Patient | 86 | Female | 101.2 | 1.58 | 40.5 |
| 129 | Patient | 78 | Male | 64.5 | 1.63 | 24.3 |
| 130 | Patient | 64 | Male | 54.7 | 1.63 | 20.6 |
| 131 | Patient | 62 | Female | 80 | 1.57 | 32.5 |
| 132 | Control | 65 | Male | 57.6 | 1.67 | 20.7 |
| 133 | Control | 58 | Male | 76.3 | 1.68 | 27 |
| 134 | Control | 36 | Female | 76.8 | 1.67 | 27.5 |
| 135 | Patient | 65 | Male | 92 | 1.72 | 31.1 |
| 136 | Patient | 62 | Male | 88 | . | . |
| 137 | Patient | 65 | Female | 77.5 | 1.5 | 34.4 |
| 138 | Patient | 84 | Female | 70 | 1.55 | 29.1 |
| 139 | Patient | 50 | Female | . | . | . |
| 140 | Patient | 54 | Male | 85 | 1.67 | 30.5 |
| 141 | Patient | 63 | Male | 80 | 1.63 | 30.1 |
| 142 | Patient | 55 | Male | 70.8 | 1.6 | 27.7 |
| 143 | Patient | 54 | Female | 78 | 1.55 | 32.5 |
| 144 | Patient | 45 | Male | 79.3 | 1.77 | 25.3 |
| 145 | Patient | 46 | Female | 63.2 | 1.52 | 27.4 |
| 146 | Patient | 49 | Male | 73 | 1.64 | 27.1 |
| 147 | Patient | 74 | Female | 45 | 1.45 | 21.4 |
| 148 | Patient | 62 | Female | . | . | . |
| 149 | Patient | 38 | Male | 73.5 | 1.77 | 23.5 |
| 150 | Patient | 66 | Female | . | . | . |
| 151 | Patient | 60 | Female | 59.6 | 1.57 | 24.2 |
| 152 | Patient | 78 | Female | 65 | . | . |
| 153 | Patient | 59 | Male | . | . | . |
| 154 | Patient | 55 | Female | 65 | 1.56 | 26.7 |
| 155 | Patient | 68 | Male | . | . | . |
| 156 | Patient | 36 | Male | . | . | . |
| 157 | Patient | 42 | Male | . | . | . |
| 158 | Patient | 80 | Male | 52 | 1.52 | 22.5 |
| 159 | Patient | 43 | Male | 64 | 1.72 | 21.6 |
| 160 | Patient | 65 | Female | . | . | . |
| 161 | Patient | 55 | Female | . | . | . |
| 162 | Patient | 50 | Male | . | . | . |
| 163 | Patient | 55 | Female | . | . | . |
| 164 | Patient | 71 | Male | 90 | 1.7 | 31.1 |
| 165 | Patient | 60 | Male | 94.4 | 1.62 | 36 |
| 166 | Patient | 68 | Male | 61 | 1.58 | 24.4 |
| 167 | Patient | 64 | Male | 92 | 1.68 | 32.6 |

| | | | | Hepatitis | Hepatitis | | |
|---|---|---|---|---|---|---|---|
| Number | Cohort | TNM_T | TNM_N | B | C | HIV | |
| 1 | Patient | T3 | N2 | Negative | Negative | Negative | |
| 2 | Patient | T3 | N1 | Negative | Negative | Negative | |
| 3 | Patient | T4b | N2 | Negative | Negative | Negative | |
| 4 | Patient | Tx | Nx | Negative | Negative | Negative | |
| 5 | Control | | | Negative | Negative | Negative | |
| 6 | Control | | | Negative | Negative | Negative | |
| 7 | Control | | | Negative | Negative | Negative | |
| 8 | Patient | T4a | N0 | Negative | Negative | Negative | |
| 9 | Control | | | Negative | Negative | Negative | |
| 10 | Control | | | Negative | Negative | Negative | |
| 11 | Control | | | Negative | Negative | Negative | |
| 12 | Control | | | Negative | Negative | Negative | |
| 13 | Control | | | Negative | Negative | Negative | |
| 14 | Control | | | Negative | Negative | Negative | |
| 15 | Control | | | Negative | Negative | Negative | |
| 16 | Control | | | Negative | Negative | Negative | |
| 17 | Patient | T3 | N0 | Negative | Negative | Negative | |
| 18 | Control | | | Negative | Negative | Negative | |
| 19 | Control | | | Negative | Negative | Negative | |
| 20 | Control | | | Negative | Negative | Negative | |
| 21 | Control | | | Negative | Negative | Negative | |
| 22 | Control | | | Negative | Negative | Negative | |
| 23 | Control | | | Negative | Negative | Negative | |
| 24 | Control | | | Negative | Negative | Negative | |
| 25 | Control | | | Negative | Negative | Negative | |
| 26 | Control | | | Negative | Negative | Negative | |
| 27 | Control | | | Negative | Negative | Negative | |
| 28 | Control | | | Negative | Negative | Negative | |
| 29 | Control | | | Negative | Negative | Negative | |
| 30 | Control | | | Negative | Negative | Negative | |
| 31 | Control | | | Negative | Negative | Negative | |
| 32 | Control | | | Negative | Negative | Negative | |
| 33 | Control | | | Negative | Negative | Negative | |
| 34 | Control | | | Negative | Negative | Negative | |
| 35 | Control | | | Negative | Negative | Negative | |
| 36 | Control | | | Negative | Negative | Negative | |
| 37 | Patient | T4 | N2 | Negative | Negative | Negative | |
| 38 | Patient | T3 | N2 | Negative | Negative | Negative | |
| 39 | Patient | T3 | N2 | Negative | Negative | Negative | |
| 40 | Patient | T4 | N2 | Negative | Negative | Negative | |
| 41 | Patient | T4 | N3 | Negative | Negative | Negative | |
| 42 | Control | | | UNA | UNA | UNA | NEG AS PER DOC DATA REVIEW |
| 43 | Patient | T4 | N0 | Negative | Negative | Negative | |
| 44 | Control | | | Negative | Negative | Negative | |
| 45 | Control | | | Negative | Negative | Negative | |
| 46 | Control | | | Negative | Negative | Negative | |
| 47 | Control | | | Negative | Negative | Negative | |
| 48 | Patient | T3 | N2 | Negative | Negative | Negative | |
| 49 | Control | | | Negative | Negative | Negative | |
| 50 | Patient | T3 | N2 | Negative | Negative | Negative | |
| 51 | Control | | | Negative | Negative | Negative | |
| 52 | Control | | | Negative | Negative | Negative | |
| 53 | Control | | | Negative | Negative | Negative | |
| 54 | Control | | | Negative | Negative | Negative | |
| 55 | Control | | | Negative | Negative | Negative | |
| 56 | Control | | | Negative | Negative | Negative | |
| 57 | Control | | | Negative | Negative | Negative | |
| 58 | Control | | | Negative | Negative | Negative | |
| 59 | Control | | | Negative | Negative | Negative | |
| 60 | Control | | | Negative | Negative | Negative | |
| 61 | Control | | | Negative | Negative | Negative | |
| 62 | Control | | | Negative | Negative | Negative | |
| 63 | Control | | | Negative | Negative | Negative | |
| 64 | Control | | | Negative | Negative | Negative | |
| 65 | Control | | | Negative | Negative | Negative | |
| 66 | Control | | | Negative | Negative | Negative | |
| 67 | Control | | | Negative | Negative | Negative | |
| 68 | Control | | | Negative | Negative | Negative | |
| 69 | Control | | | Negative | Negative | Negative | |
| 70 | Control | | | Negative | Negative | Negative | |
| 71 | Control | | | Negative | Negative | Negative | |
| 72 | Control | | | Negative | Negative | Negative | |
| 73 | Control | | | Negative | Negative | Negative | |
| 74 | Control | | | Negative | Negative | Negative | |
| 75 | Control | | | Negative | Negative | Negative | |
| 76 | Control | | | Negative | Negative | Negative | |
| 77 | Control | | | Negative | Negative | Negative | |
| 78 | Control | | | Negative | Negative | Negative | |
| 79 | Control | | | Negative | Negative | Negative | |
| 80 | Control | | | Negative | Negative | Negative | |
| 81 | Control | | | Negative | Negative | Negative | |
| 82 | Patient | T4b | N2 | Negative | Negative | Negative | |
| 83 | Control | | | Negative | Negative | Negative | |
| 84 | Control | | | Negative | Negative | Negative | |
| 85 | Control | | | Negative | Negative | Negative | |
| 86 | Control | | | Negative | Negative | Negative | |
| 87 | Control | | | Negative | Negative | Negative | |
| 88 | Control | | | Negative | Negative | Negative | |
| 89 | Control | | | Negative | Negative | Negative | |
| 90 | Control | | | Negative | Negative | Negative | |
| 91 | Control | | | Negative | Negative | Negative | |
| 92 | Control | | | Negative | Negative | Negative | |
| 93 | Control | | | Negative | Negative | Negative | |
| 94 | Control | | | Negative | Negative | Negative | |
| 95 | Control | | | Negative | Negative | Negative | |
| 96 | Control | | | Negative | Negative | Negative | |
| 97 | Control | | | Negative | Negative | Negative | |
| 98 | Control | | | Negative | Negative | Negative | |
| 99 | Control | | | Negative | Negative | Negative | |
| 100 | Control | | | Negative | Negative | Negative | |
| 101 | Control | | | Negative | Negative | Negative | |
| 102 | Control | | | Negative | Negative | Negative | |
| 103 | Patient | T3 | N0 | Negative | Negative | Negative | |
| 104 | Control | | | Negative | Negative | Negative | |
| 105 | Control | | | Negative | Negative | Negative | |
| 106 | Control | | | Negative | Negative | Negative | |
| 107 | Control | | | Negative | Negative | Negative | |
| 108 | Patient | T4a | N0 | Negative | Negative | Negative | |
| 109 | Patient | Tx | Nx | Negative | Negative | Negative | |
| 110 | Control | | | Negative | Negative | Negative | |
| 111 | Control | | | Negative | Negative | Negative | |
| 112 | Control | | | Negative | Negative | Negative | |
| 113 | Control | | | Negative | Negative | Negative | |
| 114 | Patient | T4b | N2 | Negative | Negative | Negative | |
| 115 | Patient | T2 | N1 | Negative | Negative | Negative | |
| 116 | Patient | T3 | N1 | Negative | Negative | Negative | |
| 117 | Patient | T3 | N1 | Negative | Negative | Negative | |
| 118 | Patient | T2 | N2 | Negative | Negative | Negative | |
| 119 | Patient | T2 | N2 | Negative | Negative | Negative | |
| 120 | Patient | T3 | N2 | Negative | Negative | Negative | |
| 121 | Patient | T3 | N1 | Negative | Negative | Negative | |
| 122 | Patient | T3 | N0 | Negative | Negative | Negative | |
| 123 | Patient | T4b | N2 | Negative | Negative | Negative | |
| 124 | Control | | | Negative | Negative | Negative | |
| 125 | Patient | T1 | N0 | Negative | Negative | Negative | |
| 126 | Control | | | Negative | Negative | Negative | |
| 127 | Control | | | Negative | Negative | Negative | |
| 128 | Patient | T3 | N0 | Negative | Negative | Negative | |
| 129 | Patient | T2 | N0 | Negative | Negative | Negative | |
| 130 | Patient | T3 | N2 | Negative | Negative | Negative | |
| 131 | Patient | T3 | N0 | Negative | Negative | Negative | |
| 132 | Control | | | Negative | Negative | Negative | |
| 133 | Control | | | Negative | Negative | Negative | |
| 134 | Control | | | Negative | Negative | Negative | |
| 135 | Patient | T3 | N0 | Negative | Negative | Negative | |
| 136 | Patient | T3 | N2 | Negative | Negative | Negative | |
| 137 | Patient | T2 | N0 | Negative | Negative | Negative | |
| 138 | Patient | T3 | N0 | Negative | Negative | Negative | |
| 139 | Patient | T3 | N1 | Negative | Negative | Negative | |
| 140 | Patient | T3 | N0 | Negative | Negative | Negative | |
| 141 | Patient | T3 | N0 | Negative | Negative | Negative | |
| 142 | Patient | T2 | N2 | Negative | Negative | Negative | |
| 143 | Patient | T2 | N1 | Negative | Negative | Negative | |
| 144 | Patient | T3 | N2 | Negative | Negative | Negative | |
| 145 | Patient | T3 | N2 | Negative | Negative | Negative | |
| 146 | Patient | T3 | N1 | Negative | Negative | Negative | |
| 147 | Patient | T3 | N2 | Negative | Negative | Negative | |
| 148 | Patient | T3 | N0 | Negative | Negative | Negative | |
| 149 | Patient | T2 | N2 | Negative | Negative | Negative | |
| 150 | Patient | T3 | N2 | Negative | Negative | Negative | |
| 151 | Patient | T4b | N2 | Negative | Negative | Negative | |
| 152 | Patient | T3 | N2 | Negative | Negative | Negative | |
| 153 | Patient | T2 | N1 | Negative | Negative | Negative | |
| 154 | Patient | T3 | N1 | Negative | Negative | Negative | |
| 155 | Patient | T3 | N2 | Negative | Negative | UNA | NEG AS PER DB2025 MAY 22 |
| 156 | Patient | T4 | N1 | Negative | Negative | Negative | |
| 157 | Patient | T4 | N0 | Negative | Negative | Negative | |
| 158 | Patient | T4 | N2 | Negative | Negative | Negative | |
| 159 | Patient | T3 | N3 | Negative | Negative | Negative | |
| 160 | Patient | T4 | N2 | Negative | Negative | Negative | |
| 161 | Patient | T4 | N1 | Negative | Negative | Negative | |
| 162 | Patient | T3 | N2 | Negative | Negative | Negative | |
| 163 | Patient | T4 | N2 | Negative | Negative | Negative | |
| 164 | Patient | T3 | N2 | Negative | Negative | Negative | |
| 165 | Patient | T3 | N1 | Negative | Negative | Negative | |
| 166 | Patient | T4b | N2 | Negative | Negative | Negative | |
| 167 | Patient | T3 | N1 | Negative | Negative | Negative | |

1. A method of diagnosing or monitoring a disease in a subject, the method comprising:
1) generating a spectroscopic profile of a sample obtained from the subject;
2) comparing the spectroscopic profile to one or more reference spectroscopic profiles to obtain a general score;
3) determining a status of the disease by comparing the general score to a threshold value.

2. The method of claim 1, wherein generating the spectroscopic profile comprises:
obtaining a raw vector, Vr, the raw vector comprising a two-dimensional vector of a plurality of wave numbers and a plurality of intensity values corresponding to the plurality of wave numbers;
filtering the raw vector; and
normalizing the plurality of intensity values of the raw vector to generate a normalized vector, Vn.

3. The method of claim 2, wherein the filtering comprises smoothing the raw vector and removing background noise.

4. The method of claim 2, wherein the spectroscopic profile is further generated by selecting a subset vector (Vf), of the normalized vector, Vn.

5. The method of claim 4, wherein the subset vector comprises between 6 and 18 components.

6. The method of claim 5, wherein the wave numbers of the subset vector are in non-overlapping ranges of between 20 to 60 cm⁻¹ in width.

7. The method of claim 4, wherein the general score is determined by a polynomial of a degree between 1 and 6 on the subset vector.

8. (canceled)

9. The method of claim 1, wherein the threshold value is tuned to a higher value for diagnosing the disease or a lower value for monitoring the disease.

10. The method of claim 5, wherein Vf comprises one or more values within a range of about 300 to about 1900 cm⁻¹.

11. The method of claim 10, wherein Vf comprises four values between about 620 to about 670 cm⁻¹, two values between about 720 to about 760 cm⁻¹, two values between about 1550 to about 1580 cm⁻¹, and four values between about 1740 to about 1790 cm⁻¹.

12. The method of claim 1, wherein the spectroscopic profile is generated via vibrational spectroscopy, field-resolved spectroscopy, frequency-resolved spectroscopy, Fourier-transform infrared spectroscopy, Raman spectroscopy, infrared attenuated total reflectance, diffuse reflectance spectroscopy, or combinations thereof.

13. (canceled)

14. (canceled)

15. (canceled)

16. The method of claim 12, wherein the spectroscopic profile is measured between about 15,000 cm⁻¹ to about 200 cm⁻¹.

17. (canceled)

18. (canceled)

19. (canceled)

20. The method of claim 1, wherein the disease is cancer.

21. The method of claim 20, wherein the cancer is breast cancer or colon cancer.

22. The method of claim 20, wherein the status is a TNM stage.

23. (canceled)

24. (canceled)

25. (canceled)

26. (canceled)

27. (canceled)

28. The method of claim 1, wherein the one or more reference spectroscopic profiles is from the subject.

29. The method of claim 1, wherein the subject is a human.

30. The method of claim 1, wherein the sample is in vitro.

31. The method of claim 1, wherein the sample comprises blood, spittle/saliva, serum, plasma, urine, sputum, sweat, semen, synovial fluids, lymphatic fluids, cerebrospinal fluids, biopsy, stool, or combinations thereof.

32. The method of claim 1, wherein the subject is asymptomatic of the disease.

33. (canceled)

34. (canceled)

35. (canceled)

36. (canceled)

37. (canceled)
